(19) United States
(12) Patent Application Publication
(10) Pub. No.: US 2003/0096781 A1
(43) Pub. Date: May 22, 2003

(54) IL-8 is an autocrine growth factor and a surrogate marker for Kaposi's sarcoma

(75) Inventors: Rizwan Masood, Walnut, CA (US); Parkash S. Gill, Agoura, CA (US)

Correspondence Address: Chris J. Ullsperger, Ph.D., Bingham McCutchen LLP, Suite 1800, Three Embarcadero Center, San Francisco, CA, 94111, US

(73) Assignee: University of Southern California, Los Angeles, CA

(21) Appl. No.: 10/232,506

(22) Filed: Aug. 30, 2002

Related U.S. Application Data
(60) Provisional application No. 60/316,666, filed on Aug. 31, 2001.

(51) Int. Cl. A61K 48/00; C12Q 1/68; C07H 21/04; C12N 5/00

(52) U.S. Cl. 514/44; 435/6; 435/375; 536/23.2

(57) ABSTRACT

Methods for treating and diagnosing Kaposi's sarcoma are provided. In one embodiment the invention provides a method of treating disease wherein the method comprises modulation of IL-8. The disease to be treated may be a disease such as Kaposi's sarcoma. In one embodiment, the invention comprises administering a therapeutic composition comprising IL-8 antisense oligonucleotides. The invention also provides a method of diagnosing Kaposi's sarcoma wherein the method comprises measuring the expression level of IL-8.

### STATEMENT REGARDING FEDERALLY SPONSORED RESEARCH

This invention was made in part with government support by Grant CA79281 awarded by the National Institute of Health. The United States Government has certain rights in the invention.

### REFERENCE TO RELATED APPLICATION

This application claims the benefit under 35 U.S.C. §19(e) of U.S. Provisional Application No. 60/316,666 filed Aug. 31, 2001, the contents of which are incorporated herein by reference.

### BACKGROUND OF THE INVENTION

Throughout this disclosure, various publications are referenced either by name, or by an Arabic numeral within parenthesis. The complete bibliographic citation for each reference is found in the specification, immediately preceding the claims. The contents of these publications are hereby incorporated by reference into the present disclosure to more fully describe the state of the art to which this invention pertains.

Kaposi's sarcoma (KS) is the most common tumor seen in patients with HIV-1 infection (Lifson A R et al. American Journal of Epidemiology 1990, 131:221-231; Reynolds P et al. American Journal of Epidemiology 1993, 137:19-30). KS causes significant morbidity and mortality through involvement of the skin and visceral organs. While the etiologic agent, if any, is unknown, substantial knowledge has been gained regarding the factors regulating the growth of tumor cells (Reynolds et al., 1993).

Kaposi's sarcoma most frequently presents as skin lesions (Lifson et al., 1990). Mucosal (oral cavity) involvement is the second most common site of disease, occurring on the palate and gums and can cause tooth loss, pain and ulceration. Lymph node involvement is common with KS. However, the precise frequency is not known due to the lack of routine lymph node biopsies.

Visceral involvement occurs frequently (in nearly 50% of the cases), especially in patients with advanced disease (Laine L et al. Arch Intern Med 1987, 147:1174-1175.). Advanced gastrointestinal (GI) KS can cause enteropathy, diarrhea, bleeding, obstruction and death. Pulmonary involvement is common and significant pulmonary KS occurs in nearly 20% of the cases (Laine L et al. Arch Intern Med 1987, 147-:1174-1175. Gill P S et al. Am J Med 1989, 87:57-61). The symptoms vary from no symptoms to dry cough, exertional dyspnea, hemoptysis and chest pain. Pulmonary function studies may show varying degree of hypoxemia. The overall survival of patients with symptomatic pulmonary KS is less than 6 months (Gill et al., 1989).

While the skin, lung, and GI tract are common sites of disease, nearly every organ can be involved with KS, including liver, spleen, pancreas, omentum, heart, pericardium, etc.

The treatment of AIDS-related Kaposi's sarcoma is palliative. Localized KS can be managed with local therapy including radiation therapy. Radiation therapy produces local toxicity and has a cumulative dose limiting toxicity. Other options for the cosmetic treatment of localized disease include cryotherapy, photodynamic therapy, intralesional vinblastine, and intralesional sclerosing agents, all of which result in local toxicity of pigmentation which may at times be worse than the lesions itself.

Both progressive KS, with local complications of pain, edema, and ulceration, and symptomatic visceral KS, require therapy which will result in rapid response. Systemic cytotoxic chemotherapy is the only treatment modality that produces rapid response. The frequency of response however depends on the agent, dose, and schedule. The response to therapy varies from 25% to over 50%. The most active agents include vinca alkaloids (vincristine, vinblastine), etoposide, anthracyclines and bleomycin. Combination therapies are more active than single agent treatments. However, the majority of cytotoxic agents cannot be administered for a prolonged period of time due to cumulative toxicity. Treatment with cytotoxic chemotherapy is palliative and the nearly all patients relapse within weeks of discontinuation of therapy.

Thus, a need exists for a rapid treatment modality for KS without the associated cumulative toxicity. This invention satisfies this need and provides related advantages as well.

### SUMMARY OF THE INVENTION

Applicants discovered that IL-8 and IL-8 receptors are expressed in KS cells and KS primary tumor tissue. Secondly, inhibition of IL-8 leads to inhibition of KS cell and tumor growth. Thirdly, supernatant of KS cells exert a mitogenic effect on endothelial cells and induce angiogenesis both of which are blocked by IL-8 neutralizing antibodies. In addition, serum IL-8 levels are significantly elevated in patients with KS compared to controls, and thus serve as a surrogate marker. Modulation of IL-8 is thus important in KS especially when combined with VEGF inhibitors.

Therefore, in one embodiment the invention provides a method of treating disease wherein the method comprises modulation of IL-8. The disease to be treated may be a disease such as Kaposi's sarcoma. In one embodiment, the invention comprises administering a therapeutic composition comprising a compound that inhibits the expression of IL-8. In one embodiment the composition comprises an IL-8 antisense oligonucleotide. In another embodiment, the composition comprises both an IL-8 antisense oligonucleotide and an agent which inhibits the expression of VEGF, such as a VEGF antisense oligonucleotide. In another embodiment, the composition comprises an agent which inhibits the expression of a growth factor which upregulates IL-8 expression, such as IL-1β.

In a related embodiment, the invention provides a method for inhibiting the activity of IL-8 comprising neutralizing IL-8 protein with antibodies specific for IL-8. In a related embodiment, the composition further comprises antibodies specific for VEGF or IL1β or both VEGF and IL1β.

The invention also provides a method of diagnosing Kaposi's sarcoma or other diseases associated with IL-8 overexpression, wherein the method comprises measuring the expression levels of IL-8. In one embodiment the method comprises detecting the expression level of IL-8 with antibodies specific for IL-8. In a related embodiment, the level of IL-8 in the serum is detected. In another embodiment, the method comprises detecting the expression level of IL-8 with labeled nucleotides which are complementary to IL-8 mRNA. In yet another embodiment, the invention comprises detecting the overexpression of KS-related molecules such as VEGF and CXCR1.

The invention also provides for kits which comprise agents useful for practicing the methods of the invention.

### BRIEF DESCRIPTION OF THE FIGURES

FIGS. 1A and 1B show that IL-8 is expressed in AIDS-KS cells. FIG. 1A is a Northern blot of total RNA (15 μg) from several AIDS-KS cell lines, Human umbilical vein endothelial cells (HUVEC), Human Aortic Smooth Muscle (AoSM) and T1 human fibroblasts. AIDS-KS cell lines are indicated as KS Y-1, KSC10, KSC29, KSC59, KSC13, and KS-38. Shown is the same membranes hybridized to the human IL-8 probe, stripped and re-probed with the β-actin probe. The β-actin signal shows integrity of RNA and equivalent loading of samples. FIG. 1B shows IL-8 protein levels in 48-hour supernatants from equal numbers of KS Y-1, KS-SLK, KSC10, KSC13, KSC59, 38-10 and T1 cells cultured in RPMI and 10% FCS. Supernatants were analyzed for IL-8 protein by ELISA.

FIGS. 2A and 2B show that IL-8 receptors α and β (CXCR-1 and CXCR-2) are expressed in AIDS-KS. FIG. 2A shows that immunohistochemical analysis of CXCR1 and CXCR2 expression in an AIDS-KS cutaneous lesion and KS cells in culture. The left hand panel depicts a representative AIDS-KS biopsy stained with isotype-matched control (top), antibody to CXCR1 (middle) and CXCR2 (bottom). Distinct staining for CXCR1 is observed as brown color, no staining was observed for CXCR2 or in the control panel. The right hand panel shows cultured KS cells (KS Y-1) stained in the same manner. Strong staining (red color) for CXCR1 is observed in this case with weaker staining for CXCR2. Isotype control was negative. FIG. 2B shows expression of CXCR1 and CXCR2 in KS Y-1, KS-SLK, KSC59 and HUVECs. Total RNA was extracted from 3×10⁶ cells. Equal amounts of total RNA were reverse transcribed to generate cDNA that was subjected to either CXCR1 or CXCR2 PCR amplification using a set of primers as described in Materials and Methods. The amplified products, along with size markers (M), were electrophoresed on 0.7% agarose gel containing ethidium bromide and photographed. RT-PCR for β-actin is included to show the equivalence and integrity of the input RNA. Specific bands for CXCR1 and CXCR2 are seen in all cells tested, with a relatively lower level expression of both receptors in HUVECs.

FIG. 3 shows the effect of growth factors on IL-8 production. Equal numbers of KS Y-1 cells were plated in 24 plates and treated with various test compounds; hydrocortisone (HC), IL-1β, Oncostatin-M (Onco-M), IL-6 and IL-4 for 24 hr. Supernatants were collected, centrifuged to remove cell debris and tested for IL-8 using ELISA. Results are shown as mean±standard deviation of triplicate wells.

FIGS. 4A-D show the effect of IL-8-S and IL-8-AS oligonucleotides on the viability of KS cells. FIG. 4A shows the effect of IL-8 S and IL-8 AS oligonucleotides on viability of KS cells. For the experiments shown in FIG. 4A, KSC-59 and KS-SLK cells were seeded at equal numbers (1×10⁴/well). Cells were treated with IL-8 S or IL-8 AS at concentrations ranging from 1 to 10 μM on day 2 and 4. Cell viability was measured by MTT on day 5.

FIG. 4B shows the effect of IL-8 S and IL-8 AS oligonucleotides on IL-8 gene expression. Total RNA was isolated from AIDS-KS cells treated with various concentrations of IL-8 S and IL-8 AS for 24 hr, and subjected to semiquantitative RT-PCR using IL-8 specific primers. Samples of the PCR reactions were removed at cycles 20, 25, 30 and 35, and fractionated on 1% agarose. The 311 bp PCR product of IL-8 was observed in all reactions. Band intensity was quantititated using FirstQuant software from BioRad. Shown is the histogram of band intensities from the exponential phase of the PCR reactions (cycle 20).

FIG. 4C shows that IL-8 AS inhibits the production of IL-8 protein in KS-SLK cells. 1×10⁴ cells were treated for 24 h with oligonucleotides after which the supernatants were collected. IL-8 protein was determined by ELISA (R&D Systems). Shown are the mean and standard deviations of duplicate experiments.

FIG. 4D shows that neutralizing monoclonal antibody to IL-8 reduces KS-SLK cell viability. 1×10⁴ cells were plated and treated on days 1 and 2 with neutralizing monoclonal antibody to human IL-8 (clone 6217.111) or, as control, perforin antibody. Cell viability was measured by MTT on day 3. Shown are the mean and standard deviations duplicated determinations.

FIG. 5 shows the effect of KS supernatants, IL-8 antibody and IL-10 antibody on the growth of HUVECs. HUVECs (1×10⁴) were seeded in 24 wells plates using endothelial cell growth media (Clonetics, San Diego) and allowed to attach overnight. The growth media was changed to 10% RPMI media without any growth factors for the next four hours. This medium was then removed and replaced with KS-SLK conditioned medium (CM) concentrated either five or ten fold, with or without IL-8 and IL-10 antibody. Cell proliferation was measured after 48 hours. The data represent the mean±standard deviation of three experiments performed in quadruplicate.

FIG. 6 shows that IL-8 produced by KS cells supports angiogenesis in CAMs. CAMs of ten days old chicken embryos were treated with filter discs saturated with five or ten fold concentrated KS cell conditioned medium (CM), with or without IL-8 antibody (1 μg/disc). Positive controls for angiogenesis were discs saturated with VEGF (400 ng/disc) in PBS. Baseline control consisted of filter discs saturated with PBS alone, and. Angiogenic response was measured by counting blood vessels branch points after 48 h treatment. The results represent the mean±standard deviation of three separate experiments done in six CAMs each.

FIG. 7 shows that IL-8 is required for in vivo KS tumor growth. Growth curves of subcutaneous KS-SLK tumor xenografts in. Tumors were initiated by the injection of 2×10⁶ cells in the hind flanks of 5-week old BalbC nude mice. The treatment group received anti human IL-8 monoclonal antibody (100 μg, i.p.) on days 4, 8, and 11 (indicated by arrows). Control mice received an equal volume of diluent (PBS) on the same days. Shown are the means±s.e.m. for 6 mice/group. Asterisks indicate significant differences between control and treated groups as determined by Student's T-test (P>0.05).

### DETAILED DESCRIPTION OF THE INVENTION

The present invention employs oligomeric antisense compounds, particularly oligonucleotides, for use in modulating the function of nucleic acid molecules encoding IL-8, ultimately modulating the amount of IL-8 produced. This is accomplished by providing antisense compounds which specifically hybridize with one or more target nucleic acids encoding IL-8.

IL-8 receptors belong to the superfamily of seven transmembrane, G protein-coupled receptors. Two types of IL-8 receptors have been reported IL-8R-A and IL-8R-B also called as CXCR1 and CXCR2 respectively. These two receptors share 77% amino acid identity. IL-8 receptors are found on neutrophils, basophils, T-lymphocytes, monocytes, and keratinocytes. Both receptors are also expressed on endothelial cells (43).

IL-8 is a member of a family of related proinflammatory cytokines that has a molecular mass of about 10-kDa. IL-8 is synthesized and secreted by LPS-stimulated monocytes, macrophages and by numerous other nonleukocytic cell types, including epithelial and endothelial cells, lymphocytes and keratinocytes (35-37). IL-8 was initially identified as a chemoattractant for neutrophils but not monocytes (38) and has been shown to activate neutrophils in inflammatory sites (39). IL-8 has also been shown to induce angiogenesis (40). In nude mice xenografts IL-8 expression correlates directly with metastatic potential in a variety of tumors (41). However, the expression of IL-8 depends on the organ microenvironment (42).

The invention provides compounds directed at inhibiting the expression or activity of IL-8. In one embodiment, the invention provides an antisense compound 8 to 50 nucleobases in length targeted to a nucleic acid molecule encoding IL-8, wherein said antisense compound comprises at least an 8 nucleobase portion of the sequence 5′-GTT GGC GCA GTG TGG TCC ACT CTC AAT CAC-3′ and wherein said compound inhibits IL-8. In a preferred embodiment, the compound is an antisense oligonucleotide which inhibits IL-8 in vivo or in vitro. Preferably, the antisense oligonucleotides of the invention inhibit the proliferation of cultured Kaposi's sarcoma cells at an IC₅₀ of less than or equal to about 5 μM.

The invention also provides for modified oligonucleotides. In a preferred embodiment, the antisense oligonucleotides of the invention comprise at least one modified internucleoside linkage, preferably a phosphorothioate linkage.

The invention also provides compositions comprising antisense compounds and a carrier or diluent. In one embodiment, the invention provides compositions which comprise one or more antisense compounds, such as an antisense oligonucleotide, and a colloidal dispersion system.

The invention also provides a method of inhibiting the expression of IL-8 in cells or tissues, including Kaposi's sarcoma cells and Kaposi's sarcoma tumor tissue, in vitro or in an animal, comprising the step of contacting the cells or tissues with an agent that inhibits IL-8 expression. Such agents include the antisense compounds of the invention (e.g., the antisense oligonucleotide 5′-GTT GGC GCA GTG TGG TCC ACT CTC AAT CAC-3′), an IL-8 antibody, and an agent that inhibits the activity of IL-1β (e.g., an IL-1β antibody). In another embodiment of the invention, the method further comprises an antagonist of VEGF.

The invention also provides a method for determining the likelihood of Kaposi's sarcoma in a subject comprising comparing the level of IL-8 obtained from a subject sample to the level of IL-8 in a control sample, whereby a positive diagnosis is made when said level from a subject sample is greater than said control. In a further embodiment, the invention provides a method for monitoring the progression of Kaposi's sarcoma in a subject, comprising performing two or more comparisons between the concentration of IL-8 obtained from a suitable subject sample to a control IL-8 concentration, whereby the progression of Kaposi's sarcoma is relative to the concentration of said IL-8 concentration in said subject. The invention provides methods wherein either IL-8 protein (e.g., in cells, tissue, or serum) or IL-8 mRNA may be detected.

In another embodiment, the invention provides a method for determining the likelihood of Kaposi's sarcoma in a subject comprising comparing the level of VEGF obtained from a serum sample of a subject to the level of VEGF of a control, whereby the likelihood of Kaposi's sarcoma is greater when said concentration is greater than said control. In a related embodiment, the invention provides a method for monitoring the progression of Kaposi's sarcoma in a subject, comprising performing two or more comparisons between the level of VEGF obtained from a serum sample of a subject to the level of VEGF in a control sample, whereby an increase in concentration is associated with progression of Kaposi's sarcoma in said subject. In another embodiment, the invention provides method for assesssing the risk of Kaposi's sarcoma in a subject comprising comparing the concentration of CRCX1 mRNA or protein obtained from the subject sample to a control CRCX1 concentration, whereby a concentration of CRCX1 from the subject sample greater than the control concentration is associated with an increased risk of Kaposi's sarcoma.

In one embodiment, the invention also provides an isolated polynucleotide consisting essentially of 5′-GTT GGC GCA GTG TGG TCC ACT CTC AAT CAC-3′, as well as an isolated polynucleotide that hybridizes under stringent conditions to the sequence 5′-GTT GGC GCA GTG TGG TCC ACT CTC AAT CAC-3′. In a related embodiment, the invention provides an isolated polynucleotide that is complementary to the nucleotide sequence 5′-GTT GGC GCA GTG TGG TCC ACT CTC AAT CAC-3′, as well as an isolated polynucleotide that hybridizes under stringent conditions to this complementary polynucleotide.

In another embodiment, the invention provides a vector comprising a gene encoding an RNA consisting essentially of the sequence 5′-GUU GGC GCA GUG UGG UCC ACU CUC AAU CAC-3′, wherein said gene is operably linked to a promoter capable of expressing said RNA in a cell, e.g., a Kaposi's sarcoma cell.

The invention also provides host cells, including Kaposi's sarcoma cells, which comprise the antisense compounds and/or vectors of the invention.

In a further embodiment, the invention provides kits for detecting or monitoring the progression of Kaposi's sarcoma in a subject comprising an agent that detects the level of an agent selected from the group consisting of IL-8 and CRCX1 and instructions for use.

These embodiments and other features of the invention will become more fully apparent when the following detailed description is read in conjunction with the accompanying figures.

As used herein, the terms “target nucleic acid” and “nucleic acid encoding IL-8” encompass DNA encoding IL-8, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of IL-8. In the context of the present invention, “modulation” means either an increase (stimulation) or a decrease (inhibition) in the expression of a gene product. In the context of the present invention, inhibition is a preferred form of modulation of gene expression and mRNA is a preferred target. Further, since many genes (including IL-8) have multiple transcripts, “modulation” also includes an alteration in the ratio between gene products, such as alteration of mRNA splice products.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding IL-8. The targeting process also includes determination of a site or sites within this gene for the antisense interaction to occur such that the desired effect, e.g., detection or modulation of expression of the protein, will result.

Once one or more target sites have been identified, oligonucleotides are chosen which are sufficiently complementary to the target, i.e., hybridize sufficiently well and with sufficient specificity, to give the desired effect.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

The terms “isolated polynucleotide” or “isolated oligonucleotide” refer to polynucleotide or oligonucleotide sequences, respectively, that are substantially free from other nucleic acid sequences, such as and not limited to other chromosomal and extrachromosomal DNA and RNA. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds.

“Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, or in the case of in vitro assays, under conditions in which the assays are performed.

Estimates of effective degrees of complementarity are provided by either DNA-DNA or DNA-RNA hybridization under conditions of stringency as is well understood by those skilled in the art (Hames and Higgins, Eds. (1985) Nucleic Acid Hybridisation, IRL Press, Oxford, U.K.). Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45 degrees C. for 30 min, and then repeated twice with 0.2×SSC, 0.5% SDS at 50 degrees C. for 30 min. A more preferred set of stringent conditions uses higher temperatures in which the washes are identical to those above except for the temperature of the final two 30 min washes in 0.2×SSC, 0.5% SDS which was increased to 60 degrees C. Another preferred set of highly stringent conditions uses two final washes in 0.1×SSC, 0.1% SDS at 65 degrees C.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases. Particularly preferred are antisense oligonucleotides comprising from about 8 to about 30 nucleobases (i.e. from about 8 to about 30 linked nucleosides). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′-, 3′- or 5′-hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure. However, open linear structures are generally preferred. Within the oligonucleotide structure, the phosphate groups are commonly referred to as forming the internucleoside backbone of the oligonucleotide. The normal linkage or backbone of RNA and DNA is a 3′-5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553-6556), cholic acid (Manoharan et al., Bioorg., Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

In another related embodiment, compositions of the invention may contain one or more antisense compounds, particularly oligonucleotides, targeted to a first nucleic acid and one or more additional antisense compounds targeted to a second nucleic acid target. Two or more combined compounds may be used together or sequentially.

As an alternative to targeted antisense delivery, targeted ribozymes may be used. The term “ribozyme” refers to an RNA-based enzyme capable of targeting and cleaving particular base sequences in both DNA and RNA. Ribozymes have specific catalytic domains that possess endonuclease activity (Kim and Cech, Proc. Natl. Acad. Sci. USA, (1987) 84: 8788-8792; Gerlach et al., Nature (1987) 328:802-805; Forster and Symons, (1987) Cell, 49: 211-220). For example, a large number of ribozymes accelerate phosphoester transfer reactions with a high degree of specificity, often cleaving only one of several phosphoesters in an oligonucleotide substrate (Cech et al., Cell, (1981) 27:487-496; Michel and Westhof, (1990) J. Mol. Biol., 216:585-610; Reinhold-Hurek and Shub, (1992) Nature, 357:173-176). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., (1991) Proc Natl Acad Sci USA, 88:10591-10595; Sarver et al., Science, (1990) 247:1222-1225). Recently, it was reported that ribozymes elicited genetic changes in some cell lines to which they were applied; the altered genes included the oncogenes H-ras, c-fos and genes of HIV. Most of this work involved the modification of a target mRNA, based on a specific mutant codon that is cleaved by a specific ribozyme.

Ribozymes can either be targeted directly to cells, in the form of RNA oligonucleotides incorporating ribozyme sequences, or introduced into the cell as an expression vector encoding the desired ribozymal RNA. Ribozymes may be used and applied in much the same way as described for antisense polynucleotide. Ribozyme sequences also may be modified in much the same way as described for antisense polynucleotide. For example, one could incorporate non-Watson-Crick bases, or make mixed RNA/DNA oligonucleotides, or modify the phosphodiester backbone, or modify the 2′-hydroxy in the ribose sugar group of the RNA.

Alternatively, the antisense oligo- and polynucleotides according to the present invention may be provided as RNA via transcription from expression constructs that carry nucleic acids encoding the oligo- or polynucleotides. Throughout this application, the term “expression construct” is meant to include any type of genetic construct containing a nucleic acid encoding an antisense product in which part or all of the nucleic acid sequence is capable of being transcribed. Typical expression vectors include bacterial plasmids or phage, such as any of the pUC or Bluescrip™ plasmid series or, as discussed further below, viral vectors adapted for use in eukaryotic cells.

In preferred embodiments, the nucleic acid encodes an antisense oligo- or polynucleotide under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid encoding the inhibitory peptide is not believed to be important, so long as it is capable of expressing the peptide in the targeted cell. Thus, where a human cell is targeted, it is preferable to position the nucleic acid coding the inhibitory peptide adjacent to and under the control of a promoter that is active in the human cell. Generally speaking, such a promoter might include either a human or viral promoter.

In various embodiments, the human cytomegalovirus (CMV) immediate early gene promoter, the SV40 early promoter and the Rous sarcoma virus long terminal repeat can be used to obtain high-level expression of various proteins. The use of other viral or mammalian cellular or bacterial phage promoters which are well-known in the art to achieve expression of peptides according to the present invention is contemplated as well, provided that the levels of expression are sufficient for a given purpose.

By employing a promoter with well-known properties, the level and pattern of expression of an antisense oligo- or polynucleotide can be optimized. Further, selection of a promoter that is regulated in response to specific physiologic signals can permit inducible expression of an inhibitory protein. For example, a nucleic acid under control of the human PAI-1 promoter results in expression inducible by tumor necrosis factor. Additionally any promoter/enhancer combination (as per the Eukaryotic Promoter Data Base EPDB) also could be used to drive expression of a nucleic acid according to the present invention. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Various viral promoters, cellular promoters/enhancers, and inducible promoters/enhancers could be used in combination with the nucleic acid encoding a gene of interest in an expression construct. Additionally, any promoter/enhancer combination (as per the Eukaryotic Promoter Data Base EPDB) also could be used to drive expression of the gene. Eukaryotic cells can support cytoplasmic transcription from certain bacterial promoters if the appropriate bacterial polymerase is provided, either as part of the delivery complex or as an additional genetic expression construct.

Where a cDNA insert is employed, typically one will typically include a polyadenylation signal to effect proper polyadenylation of the gene transcript. The nature of the polyadenylation signal is not believed to be crucial to the successful practice of the invention, and any such sequence may be employed, such as human growth hormone and SV40 polyadenylation signals. Also contemplated as an element of the expression construct is a terminator. These elements can serve to enhance message levels and to minimize read through from the construct into other sequences.

In certain embodiments of the invention, the delivery of a nucleic acid in a cell may be identified in vitro or in vivo by including a marker in the expression construct. The marker would result in an identifiable change to the transfected cell permitting identification of expression. Enzymes such as herpes simplex virus thymidine kinase (tk) (eukaryotic) or chloramphenicol acetyltransferase (CAT) (prokaryotic) may be employed.

One also may include a polyadenylation signal to effect proper polyadenylation of the transcript. The nature of the polyadenylation signal is not believed to be crucial to the successful practice of the invention, and any such sequence may be employed. For example, the SV40, .beta.-globin or adenovirus polyadenylation signal may be employed. Also contemplated as an element of the expression cassette is a terminator. These elements can serve to enhance message levels and to minimize read through from the cassette into other sequences.

In another embodiment, the antisense sequences of the invention may be incorporated into a double-stranded molecule suitable for RNA interference. This method is based on the interfering properties of double-stranded RNA derived from the coding regions of genes. Termed dsRNAi, this method may be used to inhibit expression of targeted genes in mammalian cells (Bass B. (2001) Nature: 411, 428-429; Sayda M et al. (2001) Nature: 411, 494-498). A nucleotide sequence suitable for RNA interference may be directly administered to cells, or may be expressed in targeted cells by a vector with an appropriate promoter.

The term “IC50” means the concentration of a substance that is sufficient to inhibit a test parameter (such as, e.g., cell growth, tumor volume, VEGF protein expression, etc.) by about 50 percent.

The term “antagonist” means a compound that prevents the synthesis of the target molecule or binds to the cellular receptor of the target molecules or an agent that blocks the function of the target molecule.

The term “scrambled oligonucleotide” means a sequence of nucleic acids constructed so as to match the nucleic acid content but not the sequence of a specific oligonucleotide.

The term “disease or disorder” refers to a variety of diseases involving abnormal proliferation of cells, such as, for example, vascular endothelial cells. Such diseases include, but are not limited to, proliferative retinopathy (diseases of the eye in which proliferation of the blood vessels cause visual loss), macular degeneration, collagen vascular diseases, skin diseases such as psoriasis and pemphigus, diabetic retinopathy, benign tumors and cancers and precancerous conditions (e.g., premalignant cells).

The term “subject” refers to any animal, preferably a mammal, preferably a human. Veterinary uses are also intended to be encompassed by this invention.

Antisense Oligonucleotides

As described herein, the present invention provides oligonucleotide sequences that specifically inhibit the synthesis of IL-8 protein and thus are able to block cancer cell proliferation or tumor growth. In a preferred embodiment these oligonucleotides include the following sequence, IL-8-AS-1, 5′-GTT GGC GCA GTG TGG TCC ACT CTC AAT CAC-3′ (complementary to human IL-8 coding region +250 to +279).

In another embodiment the oligonucleotides sequences are modified in a variety of ways, such as mixed backbone oligonucleotides which comprise both deoxy and ribo nucleotides. Antisense oligonucleotides can also comprise truncated fragments of such sequences. Also intented to be included are the functional equivalents of these oligonucleotides.

With the published nucleic acid sequence of IL-8 (47) and this disclosure provided, those of skill in the art will be able to identify, without undue experimentation, other antisense nucleic acid sequences that inhibit IL-8 expression. For example, other sequences targeted specifically to human IL-8 nucleic acid can be selected based on their ability to be cleaved by RNAse H, or to displace the binding of the disclosed antisense oligonucleotides from a nucleic acid encoding IL-8 or a portion thereof. These oligonucleotides are preferably at least about 14 nucleotides in length, most preferably 15 to 28 nucleotides long, with 15- to 25-mers being the most common.

These oligonucleotides can be prepared by the art recognized methods such as phosphoramidite or H-phosphonate chemistry which can be carried out manually or by an automated synthesizer as described in Uhlmann et al. (Chem. Rev. (1990) 90:534-583). The oligonucleotides may be composed of ribonucleotides, deoxyribonucleotides, or a combination of both.

In one embodiment the oligonucleotides of the invention are modified to be composed of ribonucleotides and deoxyribonucleotides with the 5′ end of one nucleotide and the 3′ end of another nucleotide being covalently linked to produce mixed backbone oligonucleotides (e.g., U.S. Pat. Nos. 5,652,355; 5,264,423; 5,652,356; and 5,591,721). The mixed backbone oligonucleotides may be of varying length preferably being at least about 14 nucleotides in length, most preferably 15 to 28 nucleotides long, with 15- to 25-mers being the most common. The mixed backbone oligonucleotide may be any combination of ribonucleotides and deoxyribonucleotides. By way of example, the mixed backbone oligonucleotide may comprise a contigous stretch of deoxynucleotides (e.g., about 14 to about 8) flanked on either side by ribonucleotides (e.g., about 2 to about 4). The phosphodiester bond may be replaced with any number of chemical group such as, for example, phosphothioate.

Generally, sequences which are the functional equivalent of the antisense oligonucleotides are capable of inhibiting IL-8 as assessed in the Examples described herein. By way of example, functional equivalents of the antisense oligonucleotides disclosed herein include those with an IC₅₀ concentration of between about 2 to 5 μM, most preferably below 4 μM, in a typical KS cell proliferation assay (see Example 8). Preferably such antisense oligonucleotides are derived from the IL-8 coding region, and more preferably from the region near nucleotides +250 to +279 of the IL-8 gene. Particularly preferred are functional equivalents of the modified antisense oligonucleotides which localize in the cell nucleus without manipulation (e.g., use of cationic lipids, permeabilizing agents).

One skilled in the art will understand that the antisense nucleic acid sequences may impair the activity of a gene in a variety of ways and via interaction with a number of cellular products. Examples include, but are not limited to, the hydrolysis action catalyzed by RNAse H, the formation of triple helix structures with the duplex DNA encoding IL-8, interaction with the intron-exon junctions of pre-messenger RNA, hybridization with messenger RNA in the cytoplasm resulting in an RNA-DNA complex which is degraded by the RNAas H enzyme, or by blocking the formation of the ribosome-mRNA complex and thus blocking the translation of IL-8 protein.

Expression Levels of IL-8

The level of IL-8 expression may be measured by conventional methodology. By way of example, the level of expression of IL-8 RNA may be measured by Northern Blot Analysis, Polymerase Chain Analysis and the like (See e.g. Sambrook et al., (eds.) (1989) “Molecular Cloning, A Laboratory Manual” Cold Spring Harbor Press, Plainview, N.Y.; Ausubel et al., (eds.) (1987) “Current Protocols in Molecular Biology” John Wiley and Sons, New York, N.Y.). Likewise the level of IL-8 protein may be measured by conventional methodology, including, but not limited to, Western Blot Analysis or ELISA (See e.g. Sambrook et al., (eds.) (1989) “Molecular Cloning, A Laboratory Manual” Cold Spring Harbor Press, Plainview, N.Y.; Ausubel et al., (eds.) (1987) “Current Protocols in Molecular Biology” John Wiley and Sons, New York, N.Y.). Cell proliferation assays or cell viability assays are also well known in the art. An example of a cell proliferation assay may be found in Example 8.

IL-8 RNA levels, or IL-8 protein levels, or other indicia of IL-8 expression may also be measured at different times (i.e., sequentially) so as to monitor the progression of a disease, e.g., Kaposi's sarcoma, in a subject. Such sequential measurements may also be used to monitor the disease status of a subject who is at risk for a disease, e.g., Kaposi's sarcoma. The status of a disease state in a subject may also be determined by reference to previous measurements made in a comparable subject.

Screening Assay

A screening assay for assessing the therapeutic potential of a candidate agent, such as IL-8 antisense oligonucleotides, may be performed using cells exhibiting autocrine IL-8 growth activity (e.g., a cell line that produces and uses IL-8 for its own growth, such as KS cell lines). A variety of parameters may be used to assess the therapeutic potential of a candidate agent, as described herein.

The method of assessing the therapeutic potential of an agent to inhibit cancer cell proliferation or angiogenesis, may comprise: (i) contacting cells exhibiting autocrine growth activity with at least one candidate, and (ii) measuring the level of IL-8 expression or activity or cell growth, wherein an inhibition in IL-8 expression or cell growth is indicative of the candidate agent's therapeutic potential. The term inhibition includes a reduction, decrease, dimunition or abolition of IL-8 expression, activity or cellular proliferation. An inhibition in either IL-8 expression or cell growth also provides information relevant to determining the dosage range of the agent that may be used in vivo therapy. To determine if the level of IL-8 is altered by the candidate agent, comparison may be made to cells not exposed to the candidate agent or any other suitable control.

Any cell exhibiting IL-8 autocrine growth factor activity (e.g., those cell lines sensitive to the IL-8 antisense inhibitors of the invention) may be used in the screening assay. Preferably the cell lines are mammalian cancer cells, most preferably human cancer cells. Non-limiting examples of cancer cell lines that may be used include, but are not limited to, Kaposi Sarcoma cell lines, melanoma, pancreatic, prostate and ovarian. Alternatively, the cells used in the methods may be primary cultures (e.g., developed from biopsy or necropsy specimens). Methods of maintaining primary cell cultures or cultured cell lines are well known to those of skill in the art. Desirable cell lines are often commercially available (e.g., KSY-1 (ATCC, #CRL-11448).

To enhance the sensitivity of the screening assay, the cells may be transformed with a construct comprising nucleic acid sequences encoding the IL-8 receptor to produce cells expressing a higher level of IL-8 receptors. The nucleic acid sequences encoding the IL-8 receptor may be cDNA or genomic DNA or a fragment thereof, preferably the coding sequence used is sufficient to effect IL-8 receptor activity. Sequences for IL-8 receptors are known in the art. Vectors suitable for use in expressing the IL-8 receptor are constructed using conventional methodology (See e.g. Sambrook et al., (eds.) (1989) “Molecular Cloning, A laboratory Manual” Cold Spring Harbor Press, Plainview, N.Y. ; Ausubel et al., (eds.) (1987) “Current Protocols in Molecular Biology” John Wiley and Sons, New York, N.Y.) or are commercially available.

The means by which the cells may be transformed with the expression construct includes, but is not limited to, microinjection, electroporation, transduction, transfection, lipofection calcium phosphate particle bombardment mediated gene transfer or direct injection of nucleic acid sequences or other procedures known to one skilled in the art (Sambrook et al. (1989) in “Molecular Cloning A Laboratory Manual,” Cold Spring Harbor Press, Plainview, N.Y.). For various techniques for transforming mammalian cells, see, e.g., Keown et al. (Methods in Enzymology (1990) 185:527-537). One of skill in the art will appreciate that vectors may not be necessary for the antisense oligonucleotides applications of the subject invention. Antisense oligonucleotides may be introduced into a cell, preferably a cancer cell, by a variety of methods, including, but not limited to, liposomes or lipofection (Thierry, A. R. et al (1993) Biochem Biophys Res Commun 190:952-960; Steward, A. J. et al (1996) Biochem Pharm 51:461-469) and calcium phosphate.

Additional exemplary protocols, cell lines, and compositions which may be used for screening potential anti-IL-8 agents, or which may be modified for screening other agents of interest, are provided in the Examples section of this Application.

Other Candidate Agents

Other candidate agents suitable for assaying according to the methods of the subject application may be any type of molecule from, for example, chemical, nutritional or biological sources. The candidate agent may be a naturally occurring or synthetically produced. For example, the candidate agent may encompass numerous chemical classes, though typically they are organic molecule, preferably small organic compounds having a molecular weight of more than 50 and less than about 2,500 Daltons. Such molecules may comprise functional groups necessary for structural interaction with proteins or nucleic acids. By way of example, chemical agents may be novel, untested chemicals, agonists, antagonists, or modifications of known therapeutic agents.

The agents may also be found among biomolecules including, but not limited to, peptides, saccharides, fatty acids, antibodies, steroids, purines, pryimidines, toxins conjugated cytokines, derivatives or structural analogs thereof or a molecule manufactured to mimic the effect of a biological response modifier. Examples of agents from nutritional sources include, but is not limited to, extracts from plant or animal sources or extracts thereof. Preferred agents include antisense oligonucleotides or antibodies.

The agents may be obtained from a wide variety of sources including libraries of synthetic or natural compounds. Alternatively, libraries of natural compounds in the form of bacterial, fungal, plant, and animal extracts are available or readily produced, natural or synthetically produced libraries or compounds are readily modified through conventional chemical, physical and biochemical means, and may be used to produce combinatorial libraries. Known pharmacological agents may be subjected to random or directed chemical modifications, such as acylation, alkylation, esterification, acidification, etc. to produce structural analogs.

The candidate agents which are antagonists of IL-8 may inhibit abnormal cellular proliferation in a variety of ways. For example, the antagonist may be capable of inhibiting the production of IL-8, or interfere with the binding of IL-8 to its cognate receptors or interfere with the biological effects of IL-8. Examples include, but are not limited to, antibodies against IL-8 or its receptors, (e.g., CXCR1), soluble forms of IL-8 receptors that bind IL-8 away from the cells, or agents that inhibit transmission of IL-8 binding into the cell such as protein kinase inhibitors can also be used.

Antibodies

The present invention also provides polyclonal and/or monoclonal antibodies, including fragments and immunologic binding equivalents thereof, which are capable of specifically binding to the polynucleotide sequences of the specified gene and fragments thereof, as well as the corresponding gene products and fragments thereof. The therapeutic potential of the antibodies may be evaluated in the screening methods described herein. In general, techniques for preparing polyclonal and monoclonal antibodies as well as hybridomas capable of producing the desired antibody are well known in the art (Campbell, A. M. (1984) Monoclonal Antibodies Technology: Laboratory Techniques in Biochemistry and Molecular Biology, Elsevier Science Publishers, Amsterdam, The Netherlands; Kohler, G. and Milstein, C. (1975) Nature 256: 495-497). These include, e.g., the trioma technique and the human B-cell hybridoma technique (Kozbor, D., et al. (1983) Immunology Today 4:72; Cole et al. (1985) in Monoclonal Antibodies and Cancer Therapy, Alan R. Liss, Inc., pp. 77-96).

Antibodies may be generated using standard techniques described herein or using conventional techniques, such as described in U.S. Pat. Nos. 5,837,492; 5,800,998 and 5,891,628, against the proteins themselves or against peptides corresponding to the binding domains of the proteins. Such antibodies include but are not limited to polyclonal, monoclonal, Fab fragments, F(ab′)₂ fragments, single chain antibodies, chimeric antibodies, humanized antibodies etc.

Any animal (mouse, rabbit, etc.) that is known to produce antibodies can be immunized with the immunogenic composition. Methods for immunization are well known in the art and include subcutaneous or intraperitoneal injection of the immunogen. One skilled in the art will recognize that the amount of the protein encoded by the nucleic acids of the present invention used for immunization will vary based on the animal which is immunized, the antigenicity of the immunogen, and the site of injection. The protein which is used as an immunogen may be modified or administered in an adjuvant to increase its antigenicity. Methods of increasing antigenicity are well known in the art and include, but are not limited to, coupling the antigen with a heterologous protein (such as globulin, β-galactosidase, KLH, etc.) or through the inclusion of an adjuvant during immunization.

For monoclonal antibodies, spleen cells from the immunized animals are removed, fused with myeloma cells, such as SP2/0-Ag14 myeloma cells, and allowed to become monoclonal antibody producing hybridoma cells. Any one of a number of methods well known in the art can be used to identify hybridoma cells that produce an antibody with the desired characteristics. These include screening the hybridomas with an enzyme-linked immunosorbent assay (ELISA), western blot analysis, or radioimmunoassay (RIA) (Lutz et al., Exp. Cell Research 1988, 175:109-124). Hybridomas secreting the desired antibodies are cloned and the immunoglobulin class and subclass may be determined using procedures known in the art (Campbell, 1984).

Techniques described for the production of single chain antibodies (U.S. Pat. No. 4,946,778) can be adapted to produce single chain antibodies to the proteins of the present invention. For polyclonal antibodies, antibody-containing antisera is isolated from an immunized animal and is screened for the presence of antibodies with the desired specificity using one of the above described procedures.

Antibodies may be used in a labeled form to permit detection. Antibodies can be labeled, e.g., through the use of radioisotopes, affinity labels (such as biotin, avidin, etc.), enzymatic labels (such as horseradish peroxidase, alkaline phosphatase, etc.) fluorescent labels (such as fluorescein or rhodamine, etc.), paramagnetic atoms, etc. Procedures for accomplishing such labeling are well-known in the art, e.g., see Sternberger, L. A. et al. (1970) J. Histochem. Cytochem. 18: 315; Bayer, E. A. et al. (1979) Meth. Enzym. 62: 308; Goding, J. W. (1976) J. Immunol. Meth. 13: 215. The labeled antibodies of the present invention can then be used for in vitro, in vivo, and in situ assays to identify the cells or tissues in which a fragment of the polypeptide of interest is expressed. Preferred immunoassays are the various types of ELISAs and RIAs known in the art (Garvey, J. S. et al. (1977) Methods in Immunology, 3rd ed., W. A. Benjamin, Inc., Reading, Mass.). The antibodies themselves also may be used directly in therapies or as diagnostic reagents.

Animal Model System

The anti-IL-8 agents may be evaluated first in animal models. The safety of the compositions and methods of treatment is determined by looking for the effect of treatment on the general health of the treated animal (weight change, fever, appetite behavior etc.) monitoring of generalized toxicity, electrolyte renal and hepatic function, hematological parameters and function measurements. Pathological changes may be detected on autopsies.

Any animal based (e.g., recombinant and non-recombinant) model systems may be used to assess the in vivo efficacy of the IL-8 antisense oligonucleotides or anti-IL-8 antibodies, and to provide effective dosage ranges. For example, the relevance of the cell culture findings to the ability of an anti-IL-8 monoclonal antibody to be used for the treatment of KS was confirmed by performing experiments in vivo in a mouse model of KS (see Example 12). Tumors implanted in immunodeficient mice were treated only for a short period and the growth of the tumor was studied for several additional days. The anti-IL-8 monoclonal antibody blocked the growth of the tumor in vivo.

Diseases

The IL-8 antisense oligonucleotide or IL-8 monoclonal antibodies, or the equivalents thereof, may be used to inhibit abnormal cellular proliferation. The IL-8 antisense oligonucleotides therefore have numerous therapeutic applications in a variety of diseases including, but not limited to, diseases involving abnormal proliferation of cells, such as vascular endothelial cells (e.g., pathological angiogenesis or neovascularization). Such diseases include, but are not limited to, proliferative retinopathy (diseases of the eye in which proliferation of the blood vessels cause visual loss), macular degeneration, collagen vascular diseases, skin diseases such as psoriasis, pemphigus, diabetic retinopathy, cancers and precancerous conditions. Examples of cancer that may be treated by administration of the antisense oligonucleotides include, but are not limited to, ovarian cancer, breast cancer, pancreatic cancer, prostate cancer, melanoma, Kaposi's sarcoma, lung cancer, colon cancer, kidney cancer, prostate cancer, brain cancer, or sarcomas.

Administration of the antisense oligonucleotides, or antibodies, serve to ameliorate, attenuate or abolish the abnormal proliferation of cells in the subject. Thus, for example, in a subject afflicted with cancer, the therapeutic administration of one or more of the antisense oligonucleotides serves to attenuate or alleviate the cancer or facilitate regression of cancer in the subject. Also contemplated is administration of the antisense oligonucleotides to a subject prior to any clinical signs of disease. Examples of such individuals includes, but is not limited to, subjects with a family history of a disease such as Kaposi's sarcoma, subjects carrying a deleterious genetic mutation or subjects at risk of Kaposi's sarcoma, such as an HIV+subject.

Effective Amounts

An effective or therapeutically effective amount of the IL-8 antagonists of the invention to be administered to a subject, or functional equivalents of the antagonists, may be determined in a variety of ways. By way of example, the antisense oligonucleotides to be administered may be chosen based on their effectiveness in inhibiting the growth of cultured cancer cells for which IL-8 is an autocrine growth factor. Examples of such cell lines include, but are not necessarily limited to, Kaposi's sarcoma cell lines. By way of example, the oligonucleotides are able to inhibit the proliferation of the Kaposi's sarcoma cells at IC₅₀ concentrations between about 0.1 to about 10 μM, or between about 0.1 to about 5 μM, more preferably at less than about 4 μM. A particularly preferred technique for determining the concentration of antisense oligonucleotide capable of inhibiting proliferation of a Kaposi's sarcoma cell line is the method outlined in Example 8. An assay following such a method may be calibrated to correspond to the data provided, for example, in Table 1.

Effective concentrations of antisense oligonucleotides can be determined by a variety of techniques other than inhibition of cultured cells. For example, another suitable assay that can be used is the determination of the effect of the antisense oligonucleotide on mRNA levels in a cell, such as described in Example 8. In one embodiment, antisense oligonucleotides are capable of reducing mRNA levels for one or more forms of IL-8 by a factor of about 1.5 or more. In another embodiment, the antisense oligonucleotide is capable of reducing the mRNA levels of 2 or more forms of IL-8 by a factor of about 2 or more.

By way of example, a general range of suitable effective dosages of antisense oligonucleotides that may be used are those dosages that lead to oligonucleotide concentrations in the serum of about between about 0.5 to between about 10 μM. The daily dose may be administered in a single dose or in portions at various hours of the day. Initially, a higher dosage may be required and may be reduced over time when the optimal initial response is obtained. By way of example, treatment may be continuous for days, weeks, or years, or may be at intervals with intervening rest periods. The dosage may be modified in accordance with other treatments the individual may be receiving. However, the method of treatment is in no way limited to a particular concentration or range of the antisense oligonucleotides or functional equivalents thereof and may be varied for each individual being treated and for each derivative used.

For therapeutic purposes, one of skill in the art will appreciate that individualization of dosage may be required to achieve the maximum effect for a given individual. One skilled in the art will know the clinical parameters to evaluate to determine proper dosage for the individual being treated by the methods described herein. It is further understood by one skilled in the art that the dosage administered to a individual being treated may vary depending on the individuals age, severity or stage of the disease and response to the course of treatment. The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on the EC₅₀ (the drug concentration that provokes a response halfway between baseline and maximum) found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues.

Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years. Clinical parameters that may be assessed for determining dosage include, but are not limited to, tumor size, alteration in the level of tumor markers used in clinical testing for particular malignancies. Based on such parameters the treating physician will determine the therapeutically effective amount of antisense oligonucleotides or functional equivalents thereof to be used for a given individual. Such therapies may be administered as often as necessary and for the period of time judged necessary by the treating physician.

While it is possible for the IL-8 antagonists of the invention (or functional equivalents thereof) to be administered in a pure or substantially pure form, it is preferable to present it as a pharmaceutical composition, formulation or preparation.

Pharmaceutical Compositions

The present invention also includes pharmaceutical compositions and formulations which include the antisense compounds of the invention. The pharmaceutical compositions of the present invention may be administered in a number of ways, described herein, depending upon whether local or systemic treatment is desired and upon the area to be treated. “Treating” a disease includes preventing the onset or progression of or ameliorating the symptoms of a disease which may have already been diagnosed.

The formulations of the present invention, for both veterinary and human use, comprise one or more of the anti-IL-8 agents (e.g., an antisense oligonucleotide or a neutralizing antibody) or equivalents thereof, together with one or more pharmaceutically acceptable carriers and, optionally, other active agents or therapeutic ingredients. The carrier(s) must be “acceptable” in the sense of being compatible with the other ingredients of the formulation and not deleterious to the recipient thereof. The characteristics of the carrier will depend on the route of administration. Such a composition may contain, in addition to the one or more oligonucleotides and carrier, diluents, fillers, salts, buffers, stabilizers, solubilizers, and other materials well known in the art. The formulations may be prepared by any method well-known in the pharmaceutical art.

The pharmaceutical composition of the invention may contain other active factors and/or agents which enhance inhibition of IL-8 expression or which will reduce neovascularization. For example, combinations of synthetic oligonucleotides, each of which is directed to different regions of the IL-8 mRNA, may be used in the pharmaceutical compositions of the invention. The pharmaceutical composition of the invention may further contain other active agents such as, nucleotide analogs such as azidothymidine, dideoxycytidine, dideosyinosine, and the like or taxol or Raloxifene and the like. Such additional factors and/or agents may be included in the pharmaceutical composition to produce a synergistic effect with the synthetic oligonucleotide of the invention, or to minimize side-effects caused by the synthetic oligonucleotide of the invention. Conversely, the synthetic oligonucleotide of the invention may be included in formulations of a particular anti-IL-8 or anti-neovascularization factor and/or agent to minimize side effects of the anti-IL-8 factor and/or agent. Alternatively the methods and compositions described herein may be used as adjunct therapy.

The pharmaceutical composition of the invention may further include compounds such as cyclodextrins and the like which enhance delivery of oligonucleotides into cells, as described by Zhao et al. ((1995) Antisense Res. Dev. 5(3):185-92) or slow release polymers.

The antisense oligonucleotides the present invention can be formulated for parenteral administration, e.g., for injection via the intravenous, intramuscular, subcutaneous, intratumoral or intraperitoneal routes. The preparation of an aqueous composition that contains a antisense oligonucleotide alone or in combination with another agent as active ingredients will be known to those of skill in the art in light of the present disclosure. Typically, such compositions can be prepared as injectables, such as liquid solutions or suspensions. Solid forms, that can be formulated into solutions or suspensions upon the addition of a liquid prior to injection, as well as emulsions, can also be prepared.

When oral preparations are desired, the component may be combined with typical carriers, such as lactose, sucrose, starch, talc magnesium stearate, crystalline cellulose, methyl cellulose, carboxymethyl cellulose, glycerin, sodium alginate or gum arabic among others.

In certain cases, the formulations of the invention could also be prepared in forms suitable for topical administration, such as in creams and lotions. These forms may be used for treating skin-associated diseases, such as various sarcomas.

Additional pharmaceutical methods may be employed to control the duration of action. Controlled release preparations may be achieved through the use of polymer to complex or absorb the proteins or their derivatives. The controlled delivery may be exercised by, for example, selecting appropriate macromolecules known in the art, incorporating the one or more antisense oligonucleotides either alone or in combination with other active agents into particles of a polymeric material (e.g., polyesters, polyamino acids etc) or entrapping these materials in microcapsules prepared, for example, by coacervation techniques or by interfacial polymerization.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, e.g., Berge et al., J. of Pharma Sci., (1977) 66:1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred addition salts are acid salts such as the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embolic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic aci, 4-methylbenzenesulfoic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

Regardless of the method by which the antisense compounds of the invention are introduced into a patient, colloidal dispersion systems may be used as delivery vehicles to enhance the in vivo stability of the compounds and/or to target the compounds to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid:oligonucleotide complexes of uncharacterized structure.

In a preferred formulation, the pharmaceutical composition of the invention may be in the form of liposomes in which the synthetic oligonucleotides of the invention is combined, in addition to other pharmaceutically acceptable carriers, with amphipathic agents such as lipids which exist in aggregated form as micelles, insoluble monolayers, liquid crystals, or lamellar layers which are in aqueous solution. Suitable lipids for liposomal formulation include, without limitation, monoglycerides, diglycerides, sulfatides, lysolecithin, phospholipids, saponin, bile acids, and the like. One particularly useful lipid carrier is lipofectin. Preparation of such liposomal formulations is within the level of skill in the art, as disclosed, for example, in Szoka et al., Ann. Rev. Biophys. Bioeng. 9:467 (1980); U.S. Pat. Nos. 4,235,871, 4,501,728, 4,837,028; the text Liposomes, Marc J. Ostro, ed., Chapter 1, Marcel Dekker, Inc., New York (1983); and Hope et al., Chem. Phys. Lip. 40:89 (1986), all of which are incorporated herein by reference.

Certain embodiments of the invention provide for liposomes and other compositions containing (a) one or more antisense compounds and (b) one or more other chemotherapeutic agents which function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). See, generally, The Merck Manual of Diagnosis and Therapy, 15th Ed., Berkow et al., eds., (1987) Rahway, N.J., pp. 1206-1228. Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., (1987) Rahway, N.J., pp. 2499-2506 and 46-49, respectively. Other non-antisense chemotherapeutic agents are also within the scope of this invention. Two or more combined compounds may be used together or sequentially.

Routes of Administration

Upon formulation, solutions will be administered in a manner compatible with the dosage formulation and in a therapeutically effective amount and a variety of dosage forms. The antisense oligonucleotides formulated by the methods described herein may be delivered to the target cancer cells or any cells characterized by inappropriate cellular proliferation by a variety of methods. Examples include, but are not limited to, introducing the antisense nucleic acid of the present invention into expression vector such as a plasmid or viral expression vector. Such constructs may be introduced into a cell, preferably a cancer cell, by calcium phosphate transfection, liposome (for example, lipofectin)-mediated transfection, DEAE Dextran-mediated transfection, polybrene-mediated transfection, or electroporation. A viral expression construct may be introduced into a cell, preferably a cancer cell, in an expressible form by infection or transduction. Such viral vectors include, but are not limited to, retroviruses, adenoviruses, herpes viruses and avipox viruses. In addition, one or more oligonucleotides or vectors may be directly injected in effective amounts by a needle.

The IL-8 antagonizing compounds of the invention may be introduced by endoscopy, gene gun, or lipofection as described, for example, in the following references: Mannino, R. et al. (1988) Biotechniques, 6:682-690); Newton A. and Huestis W., Biochemistry (1988) 27:4655-4659; Tanswell, A et al. (1990) Biochmica et Biophysica Acta, 1044:269-274; and Ceccoll, J. et al. Journal of Investigative Dermatology (1989) 93:190-194).

The formulations are easily administered as the type of injectable solutions described above, with even drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intraperitoneal, oral, intercranial, cerebrospinal fluid, pleural cavity, occular, or topical (lotion on the skin) administration. In this connection, sterile aqueous media which can be employed will be known to those of skill in the art in light of the present disclosure.

By way of example, antisense nucleic acid sequences, such as antisense constructs or antisense oligonucleotides may be contacted with cancer cells in a body cavity such as, but not limited to, the gastrointestinal tract, the urinary tract, the pulmonary system or the bronchial system via direct injection with a needle or via a catheter or other delivery tube placed into the cancer cells. Any effective imaging device such as X-ray, sonogram, or fiberoptic visualization system may be used to locate the target cancer cells tissue and guide the needle or catheter tube.

Administration may also be topical (including ophthalmic and to mucous membranes including vaginal and rectal delivery), pulmonary, e.g., by inhalation or insufflation of powders or aerosols, including by nebulizer; intratracheal, intranasal, epidermal, intradermal and transdermal), oral or parenteral. Parenteral administration includes intravenous, intraarterial, subcutaneous, intraperitoneal or intramuscular injection or infusion; or intracranial, e.g., intrathecal or intraventricular, administration. oligonucleotides with at least one 2′-O-methoxyethyl modification are believed to be particularly useful for oral administration.

Alternatively, the antisense nucleic acids may be administered systemically (e.g., blood circulation, lymph system) to target cancer cells which may not be directly reached or anatomically isolated.

Kit/Drug Delivery System

All the essential materials for inhibiting IL-8 expression or for inhibiting inappropriate cellular proliferation, such as tumor cell proliferation, or angiogenesis may be assembled in a kit or drug delivery system. One or more of the antisense oligonucleotides, optionally in combination with other agents (e.g., chemotherapeutics, cytokines, antibodies directed against IL-8 etc) may be formulated into a single formulation or separate formulations. The kits may further comprise, or be packaged with, an instrument for assisting with the administration or placement of the formulation to a subject. Such an instrument may be an inhalant, syringe, pipette, forceps, measured spoon, eye dropper or any such medically approved delivery vehicle. Alternatively, the container means for the formulation may itself be an inhalant, syringe, pipette, eye dropper, or other like apparatus, from which the formulation may be administered or applied to the subject or mixed with the other components of the kit.

The components of the kit may be formulated in a variety of ways. For example, the components of the kit may be provided in one or more liquid solutions, the liquid solution preferably is an aqueous solution, with a sterile, aqueous solution being particularly preferred. The components of these kits may also be provided in dried or lyophilized forms. When reagents or components are provided as a dried form, reconstitution generally is by the addition of a suitable solvent, which may also be provided in another container means. In a preferred embodiment, the oligonucleotides of the invention may be formulated as liposomes by methods known in the art, as described herein.

The kits of the invention may also include an instruction sheet defining administration of the antisense oligonucleotides. The kits of the present invention also will typically include a means for containing the vials in close confinement for commercial sale such as, e.g., injection or blow-molded plastic containers into which the desired vials are retained. Other instrumentation includes devices that permit the reading or monitoring of reactions.

All books, articles, or patents referenced herein are incorporated by reference. The following examples illustrate various aspects of the invention, but in no way are intended to limit the scope thereof.

### EXAMPLES

Materials and Methods

Cells: KS cell lines were grown on gelatin coated plates in culture medium containing RPMI 1640, 2% Fetal calf serum (FCS), 1% sodium pyruvate, 1% essential amino acids, 1% non essential amino acids, 1 mM glutamine and 1% penicillin-streptomycin (Life Technologies, Gaithersburg, Md.). Long term spindle isolates (KSC10, KSC29, KSC-59, KSC13 and KS-38) were also established from KS lesions of AIDS-KS patient as previously described (44). These isolates have been maintained in RPMI 1640 medium supplemented with 15% FCS, 2 mM glutamine, 0.5% essential amino acids, 0.5% non essential amino acids, 1 mM sodium pyruvate, and 1% Nutridoma HU (Boehringer Mannheim, Indianapolis, Ind.) in the absence of conditioned medium from transformed T cell lines (45). KS-SLK cells (46) express endothelial cells markers (such as CD31, Tie-1, Tie-2, VEGFR-2) and vascular smooth muscle cell specific alpha-actin. KS cells have been shown to have potent angiogenic activity in chicken chorio-allantoic membranes and immunodeficient mice. Human umbilical vein endothelial cells (HUVEC) were grown on gelatin (1%) coated flasks in Iscove Modified Dulbecco's media (IMDM) and F-12 Nutrient Mixture (Ham) (1:1) media supplemented with 15% FCS, 2 mM glutamine, 30 μg/ml endothelial cell growth supplement (ECGS), (Boehringer Mannheim, Indianapolis, Ind.), 2 U/ml Heparin, 100 U/ml penicillin and 100 μg/ml streptomycin. Human Aortic Smooth Muscle (AoSM) cells (Clonetics, San Diego, Calif.) were grown in smooth muscle cell basal medium (SmBM) containing 5% FCS, 0.5 μg/ml human recombinant epidermal growth factor (EGF), 5 μg/ml insulin, 1 μg/ml human recombinant basic fibroblast growth factor (bFGF), 50 μg/ml Gentamicin and 5 ng/ml Amphotericin. Fibroblasts (T1) were grown in DMEM (Gibco, Grand Island, N.Y.) containing 10% FCS, penicillin and streptomycin. KS biopsies from several HIV patients were snap frozen and stored at −70° C. until analyzed.

IL-8 sense and antisense oligonucleotide: Phosphorothionate-modified oligonucleotides were synthesized and purified by Operon Technologies, Inc. (Alameda, Calif.), or the Core facilities at the USC/Norris Comprehensive Cancer Center, Los Angeles, Calif. IL-8 antisense (AS) oligonucleotides complementary to human IL-8 coding region (47) were synthesized. The sequence and location of this oligonucleotide is: IL-8-AS-15′-GTT GGC GCA GTG TGG TCC ACT CTC AAT CAC-3′ (nucleotides +250 to +279). An oligonucleotide consisting of the scrambled sequence S 5′-GTG ATT GAG AGT GGA CCA CAC TGC GCC AAC-3′ was used as a negative control.

Chemokines and antibodies: Human recombinant IL-1b, IL-4, IL-6, IL-8, IL-8 ELISA kits, human IL-8 monoclonal neutralizing antibody (clone 6217.111) and Oncostatin-M neutralizing polyclonal antibody were purchased from R & D Systems (Minneapolis, Minn.). Monoclonal antibodies to IL-8 receptors A and B (CXCR1 and CXCR2; clones B-1 and E-2 respectively) were purchased from Santa Cruz Biotechnology, Inc. Santa Cruz, Calif. Glucocorticoid was purchased from Sigma Chemical Company, St. Louis, Mo.

Northern Blot: Total RNA was extracted from several AIDS-KS cell lines, HUVEC, AoSM and T1 by guanidine isothiocyanate, (RNAzol: Tel-Test, Inc., Friendswood, Tex.). An aliquot (15 μg) of the total RNA from each sample was electrophoresed in a 1% agarose formaldehyde gel and transferred to nylon membranes. A probe for human IL-8 was generated by PCR amplification of the region corresponding to nucleotides −3 to +377 of the coding region of IL-8 with primers as shown in Table 1. The amplified IL-8 product was subcloned in the TA cloning vector (Invitrogen, Carlsbad, Calif.) to give pCR II™ IL-8. The IL-8 plasmid was digested with EcoR1 and the IL-8 insert was further purified using a QIAquick Gel extraction kit (Qiagen, Santa Clarita, Calif.). Radiolabeled IL-8 DNA probe was prepared using a Nick translation kit (Gibco BRL, Gaithersburg, Md.) with [α-³²P] dCTP (3000 Ci/mmol; DuPont, Boston, Mass.). RNA blots were sequentially hybridized to IL-8 and β-actin probes. The radiolabeled signal was also quantitated by using the Molecular Dynamics phosphorimager 445SI, (Sunnyvale, Calif.).

Cell Proliferation Assay: AIDS-KS cells, HUVEC and AoSM were seeded at a density of 1×10⁴ cells per well in gelatin coated 24 well plates on day 0. The cells were then treated with various concentrations of oligonucleotides ranging from 1-10 μM, or IL-8 monoclonal neutralizing antibody (10-1000 ng/mg) on days 1 and 3. Cell proliferation was measured on day 5 using 3-(4,5-dimethylthiazol-2-yl)-2,5-diphenyltetrazolium bromide (MTT) at a final concentration of 0.5 mg/ml. Cells were incubated for 2 hr, medium was aspirated, and the cells were dissolved in acidic isopropanol (90% isopropanol, 0.5% SDS and 40 mM HCl). Developed color was read in an ELISA reader at 490 nm using the isopropanol as blank (Molecular Devices, CA). The assays were performed twice in quadruplicate. These experiments were repeated in AIDS-KS cell lines with or without the addition of rhIL-8 (10 ng/ml). For HUVEC growth studies, HUVECs (2×10⁴) were seeded on gelatin coated 24 well plates in endothelial cell media as described earlier. The next day, the medium was replaced by KS culture supernatant with and without various concentration of IL-8 antibody. Cell proliferation was measured on day five.

ELISA for Human IL-8: IL-8 levels in culture supernatants were determined by a solid phase double-ligand enzyme-linked immunosorbent assay (ELISA) obtained from R & D Systems. A monoclonal antibody specific for IL-8 was pre-coated onto a microtiter plate. Standards, samples, and IL-8 conjugate to horseradish peroxidase were pipetted into the wells and any IL-8 present was sandwiched by the immobilized antibody and the enzyme-linked polyclonal antibody specific for IL-8. After removal of excess of any unbound substances and/or antibody-enzyme reagent, a substrate solution (hydrogen peroxide and chromogen tetramethylbenzidine) was added to the wells and color was developed in proportion to the amount of IL-8 bound. The color development was stopped after 30 min at room temperature and the intensity of the color was measured at 450 nm in a microplate reader (Molecular Devices, Sunnyvale, Calif.). 5

RT-PCR for IL-8 and IL-8 receptors: KS cells (1×10⁵ cells/well) were treated with various concentrations (0, 1, 5, 10 μM) of IL-8 antisense (IL-8-AS) and sense (IL-8S) oligonucleotides on day 1 and day 2. Cells were harvested on day 3 and total RNA was extracted. cDNAs were synthesized by reverse transcriptase using a random hexamer primer in a total volume of 20 μl (Superscript; Life Technologies, Gaithersburg, Md.). Five microliters of the cDNA reaction were used for PCR. Primer pairs for amplification of IL-8, CXCR1 and CXCR2 are shown in Table 1. Each PCR cycle consisted of denaturation at 94° C. for 1 min, primer annealing at 60° C. for 2 min and extension at 72° C. for 3 min. The samples were amplified for 35 cycles. Amplified products were visualized on a 1.5% agarose gel containing ethidium bromide. RT-PCR for β-actin was performed to determine the integrity and quantity of RNA.

Immunohistochemistry: Cultured KS, HUVEC and fibroblast cells were trypsinized and centrifuged on to glass slides using a Cytospin centrifuge (Shandon, Astmoor, England) and fixed in 1% paraformaldehyde in PBS for 30 min. The slides were rinsed twice in PBS and incubated with 0.2% Triton-X100 in PBS and 10% FCS for 20 min. The slides were then preincubated with 50% FCS for 30 min and again with monoclonal antibodies to CXCR1 and CXCR2 with 1:25 dilution in PBS and 10% FCS for 45 min at 37° C. After washing three times, the slides were incubated with secondary anti IgG mouse antibody. Tissue sections were also stained using the same antibodies by standard methods.

CAM Assay: Chicken allantoic membrane assay (CAM) was used to test the effect of KS cells supernatants (5×and 10×concentrates) with or without neutralizing monoclonal IL-8 antibody (1 μg/ml) on angiogenesis. Ten-day-old fertilized chicken eggs were prepared by creating a window, and filter paper discs saturated with test substances were placed on the CAM. VEGF (400 ng) was used as positive control. Concentrated KS cell supernatants alone or with neutralizing IL-8 antibody or equal amount of carrier buffer made up the test group and negative controls respectively. CAMs were harvested after 48 hr and analyzed using an Olympus stereomicroscope. The number of new branching blood vessels infiltrating under the discs were counted and photographed. Eight CAMs were studied for each test group, and the experiments were repeated twice. Blood vessels counts were performed and the results represent the mean±SD of an experiment performed in duplicate.

Serum Collection from cases with Classic KS and Controls: Cases of Classic KS (i.e., HIV negative cases) were identified from the Cancer Surveillance Program in Los Angeles County between 1989-93. For each Classic case, one neighborhood control matched on age, race, and sex was sought. Serum from twenty-three patients with Classic KS and 28 control cases were tested for various cytokines.

In vivo studies: Human KS tumor cell line KS-SLK (2×10⁶ cells) was injected subcutaneously in the lower back of 5-week old male Balb/C Nu⁺/nu⁺ athymic mice. 12 mice were randomized into two groups of 6 mice. The experimental group received IL-8 monoclonal antibody (100 μg/mouse, i.p.) on days 4, 8 and 11. Controls received and equal volume (100 μl) of PBS. The length (L) and width (W) of tumors was measured three times a week using calipers. Tumor volume was calculated with the formula V=0.52LW². Mice were sacrificed at the conclusion of the study. All mice were maintained in accord with the University of Southern California institutional guidelines governing the care of laboratory mice.

### Example 1

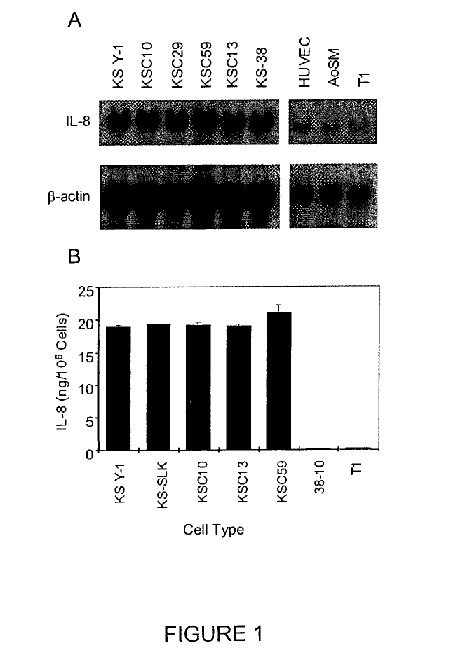

KS cell lines express high levels of IL-8: The expression of IL-8 specific mRNA in several AIDS-KS cell lines (KS Y-1, KSC10, KSC29, KSC59, KSC13 and KS-38) was examined. FIG. 1A shows that a single band for IL-8 mRNA transcripts was expressed at high levels in all AIDS-KS cell lines. HUVEC and AoSM cells also expressed a very low levels of IL-8 mRNA compared to all KS cells lines; A fibroblast cell line (T1) expressed the lowest levels of IL-8 mRNA. Supernatants from equal numbers of five KS cell lines, an HTLV-II transformed T-cell line 38-10 (45), and T1 fibroblasts were also examined for IL-8 protein levels. The levels of IL-8 protein were substantially higher in AIDS-KS cells (KS Y-1, KS-SLK, KSC-10, KSC13 and KSC59) in comparison to levels in 38-10, HUVEC, and T1 cell lines (FIG. 1B).

### Example 2

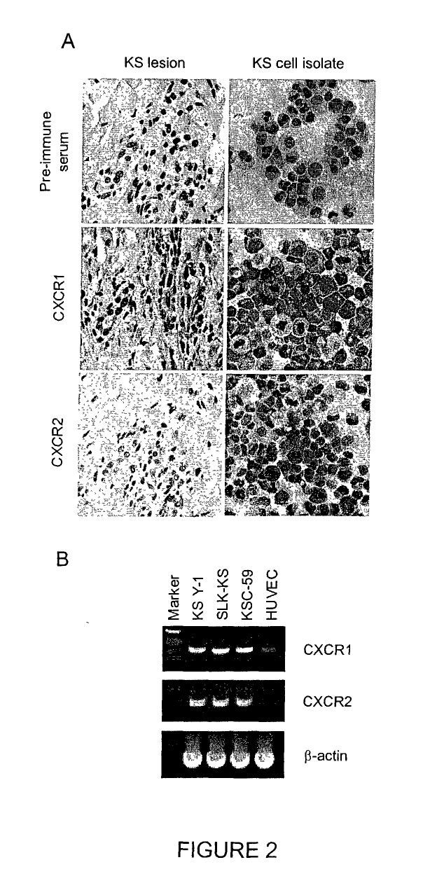

KS cells and tissues express CXCR1 and CXCR2: Expression of both IL-8 receptors was examined by immunohistochemistry in KS cell lines. A high expression of CXCR1 was observed in KS cells compared to CXCR2 as shown in FIG. 2A. Early passage of HUVECs showed a low level of both IL-8 receptors. Expression of either IL-8 receptor was undetectable in T1 fibroblasts (data not shown). Three KS cell lines, (KS Y-1, KS-SLK and KSC-59) and HUVECs were also examined for CXCR1 and CXCR2 expression by RT-PCR. All three KS cell lines showed high expression of both IL-8 receptors. HUVECs expressed both IL-8 receptors but the level of expression of both CXCR1 and CXCR2 in HUVECs was lower than in KS cells (FIG. 2B).

The expression of CXCR1 and CXCR2 in KS biopsies and skin biopsies of the same patients was also examined. Immunohistochemistry showed high levels CXCR1 in the KS biopsies from three patients. Cells expressing the receptor reveal spindle shaped morphology characteristic of the KS cells. (FIG. 2A). The skin biopsies from the same patients did not show the expression of either IL-8 receptor (data not shown). The data from cell lines was consistent with that of the primary tumor tissue, thus validating the in vitro studies.

### Example 3

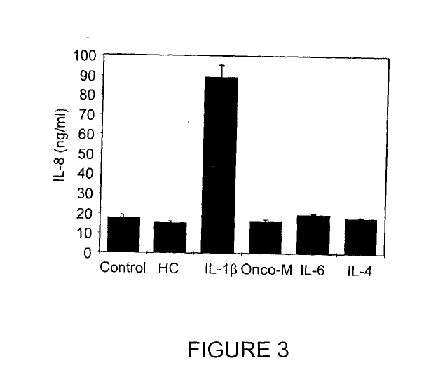

IL-1β upregulates IL-8 expression in KS cells: A number of growth factors have been shown to regulate the growth of KS cells. The role of growth factors (Oncostatin-M), cytokines (IL-1β, IL-4, IL-6), or glucocorticoids in the up-regulation of IL-8 expression in KS cells was examined. KS cells were incubated in medium alone or medium containing 10 ng/ml of either recombinant IL-1βIL-4, or IL-6 for 24 h, and culture supernatants were analyzed for IL-8 protein by ELISA. IL-1β treatment of KS cells significantly enhanced IL-8 production (4.0 fold) compared to KS cells incubated with medium alone (FIG. 3, control). KS cells treated with other growth factors (IL-4, IL-6, Oncostatin-M and Hydrocortisone) did not show a significant effect on the expression of IL-8.

### Example 4

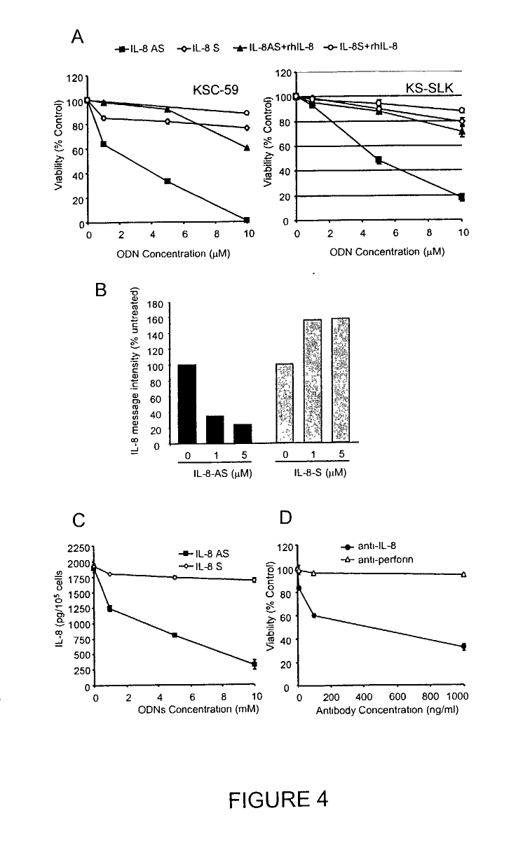

Effect of IL-8 antisense oligonucleotides on AIDS-KS cell growth IL-8 antisense oligonucleotide (IL-8 AS) corresponding to IL-8 cDNA was tested for activity against AIDS-KS cell growth at several concentrations. The growth of two KS cell lines (KSC-59, KS-SLK) was inhibited by IL-8 AS in a dose-dependent manner (FIG. 4A). The IC₅₀ of IL-8 AS oligonucleotides was 3.2 μM in KSC-59 and 4.4 μM in KS-SLK. The sense oligonucleotide (IL-8S) used as a control had minimal inhibitory effect on the growth of both KS cell lines (FIG. 4A). The effect of IL-8 AS and IL-8S on the growth of various control cell lines was also examined. No growth regulatory effects were observed on fibroblast (T1), 23-1 (B lymphoma cell line), or HUVECs with IL-8 AS (data not shown). IL-8S had no significant effect on any of the cell types examined (data not shown).

### Example 5

Exogenous rhIL-8 blocks IL-8 antisense effects on AIDS-KS cell growth: To demonstrate the specificity of the IL-8 antisense oligonucleotide on KS cell growth, KS cells were treated with IL-8 AS alone or in combination with rhIL-8. The dose-dependent inhibition of KS cell growth mediated by IL-8 antisense oligonucleotides (IL-8 AS), was blocked by addition of exogenous recombinant IL-8. These data confirm the specificity of the IL-8 antisense oligonucleotides (FIG. 4A).

### Example 6

Effect of IL-8 antisense on IL-8 mRNA level: IL-8 mRNA was also measured by RT-PCR in KS cells treated with various concentrations of IL-8 antisense and sense oligonucleotides. The PCR product of cycles ranging from 20-32 cycles was analyzed by agarose gel electrophoresis. There was a significant inhibition of IL-8 mRNA after treatment with IL-8 AS at a concentration of 5 μM in KS cells. In contrast, IL-8 S had no effect of the expression of IL-8 (FIG. 4B).

### Example 7

Effect of IL-8 antisense on IL-8 protein: IL-8 protein levels in the supernatants of KS-SLK cells treated with various concentrations of IL-8 antisense and sense oligonucleotides for 24 h were determined by ELISA. IL-8 protein decreased in a dose-dependent manner in cells treated with IL-8 AS (FIG. 4C). No appreciable effect on IL-8 was seen when the cells were treated with IL-8 S.

### Example 8

IL-8 antibody inhibits KS cell proliferation: The requirement of IL-8 as a growth factor for KS cells was confirmed using IL-8 neutralizing monoclonal antibody. Dose dependent inhibition of cell growth was observed in the KS-SLK cell line in response to incubation with IL-8 monoclonal antibody (FIG. 4D). The same line had shown growth inhibition with IL-8 AS. Unrelated polyclonal antibody (to perforin) had no effect on the viability of KS-SLK cells.

### Example 9

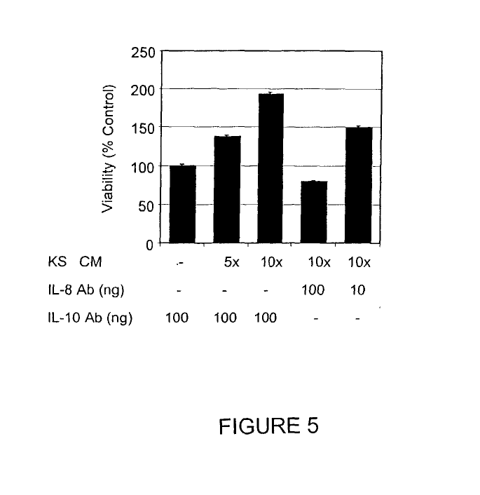

KS cell supernatant induces the growth of HUVECs: HUVECs in culture require growth supplements to survive. KS spindle cell isolates share many endothelial cell markers, yet do not require supplements for growth. We determined whether KS cells produce factors that support the growth of non-transformed HUVECs. HUVECs cultured in the presence of KS cell supernatant used as conditioned media showed a 40% increase in growth compared to control (FIG. 5). Since IL-8 is produced by the KS cells we tested whether IL-8 contributed to the growth of HUVECs by using IL-8 neutralizing antibodies. Induction of HUVEC growth was blocked by the presence of IL-8 antibody in the KS cell conditioned medium, but not by antibody to IL-10 (FIG. 5).

### Example 10

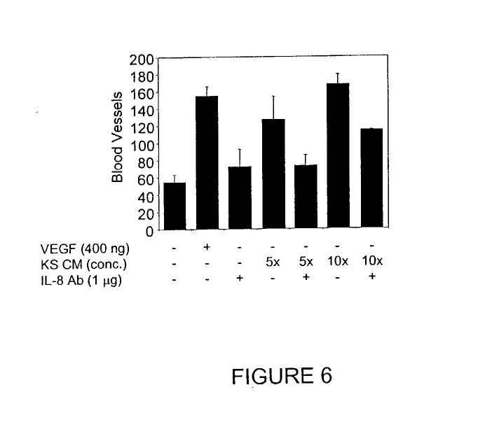

IL-8 antibody inhibits KS cells supernatant induced angiogenesis in CAM assays: In order to further investigate the angiogenic activity in KS cells supernatants CAM assays were performed. Induction of angiogenesis by KS cell supernatants was seen, and with 10×-concentrated supernatant was as effective as the positive control of 400 ng VEGF (FIG. 6). IL-8 neutralizing antibody (1 μg/disc) was able to fully block formation of blood vessels in the presence of 5×-concentrated supernatant, and partially block the effect of 10×-concentrated supernatant. This is consistent with the presence and angiogenic activity of IL-8 in the KS cell supernatants. VEGF is also produced in significant amounts by KS cells (12) and could account for the residual angiogenesis in the presence of IL-8 neutralizing antibody in the 10×KS cell supernatant.

### Example 11

Serum levels of IL-8 were higher in KS than controls: We also measured serum IL-8 levels from patients with the classic form of KS and controls. KS patients showed significantly higher levels of IL-8, mean value 142.6 pg/ml. In contrast, the levels of IL-8 in controls were significantly lower, mean value 70.8 pg/ml (Table 2). Similarly, the levels of VEGF were higher in KS than the controls, while the serum IL-6 levels were similar in both groups. These results are in agreement with the in vitro data for both VEGF and IL-8 but not IL-6. These results are the first to show that among various cellular factors associated with KS, IL-8 and VEGF are elevated in the serum in addition to locally in the tumor tissue.

### Example 12

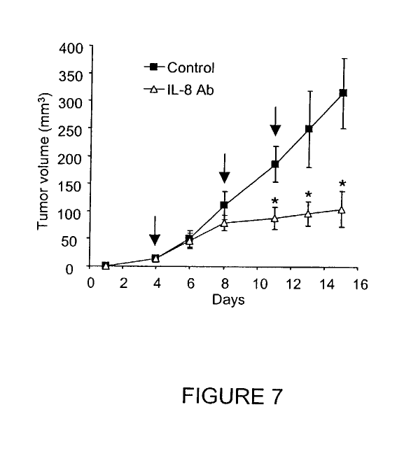

IL-8 monoclonal antibody inhibits in vivo KS tumor growth KS-SLK grown as a subcutaneous tumor xenograft in BalbC nude mice is sensitive to IL-8 ablation. The growth of established tumors was inhibited by twice-weekly treatment of the mice with neutralizing monoclonal antibody to human IL-8 (FIG. 7). After three treatments with IL-8 antibody mean tumor volume was 33% of controls, a result which was significant (P=0.027). The differences between the tumor sizes of IL-8 antibody treated mice and controls became significant after the second dose of antibody (P=0.014). These data support the in vivo requirement for IL-8 in KS tumor growth.

### Example 13

In Vivo studies of IL-8 antisense compounds. Human tumor cell lines KS Y-1, M21, and Hey (2×10⁶ cells) are injected subcutaneously in the lower back of 5-week old male Balb/C Nu⁺/nu⁺ athymic mice. In the first protocol, treatment consists of daily oral administration of IL-8-AS-1 or scrambled MBO or diluent (PBS) begun the day following tumor cell implantation and continues for two weeks. Dosing was 10 mg/kg in 100 μl PBS by gavage. In the second protocol, designed to test tumor regression, the cells are implanted and the xenograft is allowed to establish for 5 days before treatment is initiated. Treatment consists of daily intraperitoneal injection of IL-8-AS-1 (1, 5 or 10 mg/kg in a total volume of 100 μl) or diluent. Taxol (1.25 or 2.5 mg/kg) treatment, is by intraperitoneal injection on days 5 and 12. Tumor growth in mice is measured three times in a week. Mice are sacrificed at the conclusion of the study. Tumors are collected and analyzed for IL-8 levels.

It can be shown that IL-8-AS-1 specifically inhibits growth of KS Y-1 tumor xenografts in mice. The same model can be used to determine if a mixed backbone oligonucleotide functionally equivalent to IL-8-AS-1 may be made orally available. Daily oral administration of such a mixed backbone oligonucleotide (IL-8-AS-1m) over the course of two weeks is expected to result in the near complete inhibition of KS Y-1 tumor xenograft growth.

TABLE 1
Primers used to amplify IL-8 and
IL-8 receptor sequences

Size of
PCR

| | | | |
|---|---|---|---|
| | | Orien- | Product |
| Name | Primer | tation | (bp) |

| | | | |
|---|---|---|---|
| IL-8 | 5′-AACATGACTTCCAAGCTGGCCG- | Forward | 380 |
| | 3′ | | |
| | 5′-CTCTTCAAAAACTTCTCCCGACT | Reverse | |
| | CTTAAGTAT-3′ | | |
| CXCR1 | 5′-CAGTTACAGCTCTACCCTGCCC- | Forward | 964 |
| | 3′ | | |
| | 5′-GCTGTCTTTGGGCCAGGGAGTC- | Reverse | |
| | 3′ | | |
| CXCR2 | 5′-CGCCAGGCTTACCATCCAAACA- | Forward | 489 |
| | 3′ | | |
| | 5′-GGTAACACGATGACGTGCCAAG- | Reverse | |
| | 3′ | | |

TABLE 2
Serum Levels of IL-8, VEGF and IL-6 in Classical KS and Controls

| | Mean* | 95% Confiden | |
|---|---|---|---|
| | pg/ml | Interval | P-value |
| IL-8 | | | |
| Classic KS | 142.6 | 95-215 | 0.027 |
| Controls | 70.8 | 37-137 | |
| VEGF | | | |
| Classic KS | 419.9 | 337-523 | 0.019 |
| Controls | 195.6 | 112-340 | |
| IL-6 | | | |
| Classic KS | 8.5 | 6-12 | 0.41 |
| Controls | 10.0 | 7-14 | |

*Geometric Mean

### REFERENCES

1. Gill, P. S., Hamilton, A., and Naidu, Y. Epidemic (AIDS-related) Kaposi's sarcoma: Epidemiology, pathogenesis and treatment, AIDS. 7: 1-11, 1994.

2. Cai, J., Zheng, T., Lotz, M., Zhang, Y., Masood, R., and Gill, P. Glucocorticoids induce Kaposi's sarcoma cell proliferation through the regulation of transforming growth factor-beta, Blood. 89: 1491-500, 1997.

3. Ensoli, B., Nakamura, S., Salahuddin, S. Z., Biberfeld, P., Larsson, L., Beaver, B., Wong-Staal, F., and Gallo, R. C. AIDS-Kaposi's sarcoma-derived cells express cytokines with autocrine and paracrine growth effects, Science. 243: 223-6, 1989.

4. Miles, S. A., Rezai, A. R., Salazar-Gonzalez, J. F., Vander Meyden, M., Stevens, R. H., Logan, D. M., Mitsuyasu, R. T., Taga, T., Hirano, T., Kishimoto, T., and et al. AIDS Kaposi sarcoma-derived cells produce and respond to interleukin 6, Proc Natl Acad Sci U S A. 87: 4068-72, 1990.

5. Nair, B. C., DeVico, A. L., Nakamura, S., Copeland, T. D., Chen, Y., Patel, A., O'Neil, T., Oroszlan, S., Gallo, R. C., and Sarngadharan, M. G. Identification of a major growth factor for AIDS-Kaposi's sarcoma cells as oneostatin M, Science. 255: 1430-2, 1992.

6. Cai, J., Gill, P. S., Masood, R., Chandrasoma, P., Jung, B., Law, R. E., and Radka, S. F. Oncostatin-M is an autocrine growth factor in Kaposi's sarcoma, Am J Pathol. 145: 74-9, 1994.

7. Weich, H. A., Salahuddin, S. Z., Gill, P., Nakamura, S., Gallo, R. C., and Folkmann, J. AIDS-associated Kaposi's sarcoma-derived cells in long-term culture express and synthesize smooth muscle alpha-actin, Am J Pathol. 139: 1251-8, 1991.

8. Samaniego, F., Markham, P. D., Gallo, R. C., and Ensoli, B. Inflammatory cytokines induce AIDS-Kaposi's sarcoma-derived spindle cells to produce and release basic fibroblast growth factor and enhance Kaposi's sarcoma-like lesion formation in nude mice, J Immunol. 154: 3582-92, 1995.

9. Salahuddin, S. Z., Nakamura, S., Biberfeld, P., Kaplan, M. H., Markham, P. D., Larsson, L., and Gallo, R. C. Angiogenic properties of Kaposi's sarcoma-derived cells after long-term culture in vitro, Science. 242: 430-3, 1988.

10. Weindel, K., Marme, D., and Weich, H. A. AIDS-associated Kaposi's sarcoma cells in culture express vascular endothelial growth factor, Biochem Biophys Res Commun. 183: 1167-74, 1992.

11. Cornali, E., Zietz, C., Benelli, R., Weninger, W., Masiello, L., Breier, G., Tschachler, E., Albini, A., and Sturzl, M. Vascular endothelial growth factor regulates angiogenesis and vascular permeability in Kaposi's sarcoma, Am J Pathol. 149: 1851-69, 1996.

12. Masood, R., Cai, J., Zheng, T., Smith, D. L., Naidu, Y., and Gill, P. S. Vascular endothelial growth factor/vascular permeability factor is an autocrine growth factor for AIDS-Kaposi sarcoma, Proc Natl Acad Sci U S A. 94: 979-84, 1997.

13. Samaniego, F., Markham, P. D., Gendelman, R., Watanabe, Y., Kao, V., Kowalski, K., Sonnabend, J. A., Pintus, A., Gallo, R. C., and Ensoli, B. Vascular endothelial growth factor and basic fibroblast growth factor present in Kaposi's sarcoma (KS) are induced by inflammatory cytokines and synergize to promote vascular permeability and KS lesion development, Am J Pathol. 152: 1433-43, 1998.

14. Cohen, T., Nahari, D., Cerem, L. W., Neufeld, G., and Levi, B. Z. Interleukin 6 induces the expression of vascular endothelial growth factor, J Biol Chem. 271: 736-41, 1996.

15. Li, J., Perrella, M. A., Tsai, J. C., Yet, S. F., Hsieh, C. M., Yoshizumi, M., Patterson, C., Endege, W. O., Zhou, F., and Lee, M. E. Induction of vascular endothelial growth factor gene expression by interleukin-1 beta in rat aortic smooth muscle cells, J Biol Chem. 270: 308-12, 1995.

16. Ganju, R. K., Munshi, N., Nair, B. C., Liu, Z. Y., Gill, P., and Groopman, J. E. Human immunodeficiency virus tat modulates the Flk-1/KDR receptor, mitogen-activated protein kinases, and components of focal adhesion in Kaposi's sarcoma cells, J Virol. 72: 6131-7, 1998.

17. Ablashi, D., Chatlynne, L., Cooper, H., Thomas, D., Yadav, M., Norhanom, A. W., Chandana, A. K., Churdboonchart, V., Kulpradist, S. A., Patnaik, M., Liegmann, K., Masood, R., Reitz, M., Cleghorn, F., Manns, A., Levine, P. H., Rabkin, C., Biggar, R., Jensen, F., Gill, P., Jack, N., Edwards, J., Whitman, J., and Boshoff, C. Seroprevalence of human herpesvirus-8 (HHV-8) in countries of Southeast Asia compared to the USA, the Caribbean and Africa, Br J Cancer. 81: 893-7, 1999.

18. Kedes, D. H., Operskalski, E., Busch, M., Kohn, R., Flood, J., and Ganem, D. The seroepidemiology of human herpesvirus 8 (Kaposi's sarcoma-associated herpesvirus): distribution of infection in KS risk groups and evidence for sexual transmission, Nat Med. 2: 918-24, 1996.

19. Arvanitakis, L., Geras-Raaka, E., Varma, A., Gershengorn, M. C., and Cesarman, E. Human herpesvirus KSHV encodes a constitutively active G-protein-coupled receptor linked to cell proliferation [see comments], Nature. 385: 347-50, 1997.

20. Guo, H. G., Browning, P., Nicholas, J., Hayward, G. S., Tschachler, E., Jiang, Y. W., Sadowska, M., Raffeld, M., Colombini, S., Gallo, R. C., and Reitz, M. S., Jr. Characterization of a chemokine receptor-related gene in human herpesvirus 8 and its expression in Kaposi's sarcoma, Virology. 228: 371-8, 1997.

21. Cesarman, E., Nador, R. G., Bai, F., Bohenzky, R. A., Russo, J. J., Moore, P. S., Chang, Y., and Knowles, D. M. Kaposi's sarcoma-associated herpesvirus contains G protein-coupled receptor and cyclin D homologs which are expressed in Kaposi's sarcoma and malignant lymphoma, J Virol. 70: 8218-23, 1996.

22. Li, M., Lee, H., Guo, J., Neipel, F., Fleckenstein, B., Ozato, K., and Jung, J. U. Kaposi's sarcoma-associated herpesvirus viral interferon regulatory factor, J Virol. 72: 5433-40, 1998.

23. Cheng, E. H., Nicholas, J., Bellows, D. S., Hayward, G. S., Guo, H. G., Reitz, M. S., and Hardwick, J. M. A Bcl-2 homolog encoded by Kaposi sarcoma-associated virus, human herpesvirus 8, inhibits apoptosis but does not heterodimerize with Bax or Bak, Proc Natl Acad Sci U S A. 94: 690-4, 1997.

24. Sarid, R., Sato, T., Bohenzky, R. A., Russo, J. J., and Chang, Y. Kaposi's sarcoma-associated herpesvirus encodes a functional bcl-2 homologue, Nat Med. 3: 293-8, 1997.

25. Moore, P. S., Boshoff, C., Weiss, R. A., and Chang, Y. Molecular mimicry of human cytokine and cytokine response pathway genes by KSHV, Science. 274: 1739-44, 1996.

26. Nicholas, J., Ruvolo, V. R., Burns, W. H., Sandford, G., Wan, X., Ciufo, D., Hendrickson, S. B., Guo, H. G., Hayward, G. S., and Reitz, M. S. Kaposi's sarcoma-associated human herpesvirus-8 encodes homologues of macrophage inflammatory protein-1 and interleukin-6, Nat Med. 3: 287-92, 1997.

27. Kledal, T. N., Rosenkilde, M. M., Coulin, F., Simmons, G., Johnsen, A. H., Alouani, S., Power, C. A., Luttichau, H. R., Gerstoft, J., Clapham, P. R., Clark-Lewis, I., Wells, T. N.C., and Schwartz, T. W. A broad-spectrum chemokine antagonist encoded by Kaposi's sarcoma-associated herpesvirus, Science. 277: 1656-9, 1997.

28. Gao, S. J., Boshoff, C., Jayachandra, S., Weiss, R. A., Chang, Y., and Moore, P. S. KSHV ORF K9 (vIRF) is an oncogene which inhibits the interferon signaling pathway, Oncogene. 15: 1979-85, 1997.

29. Thome, M., Schneider, P., Hofmann, K., Fickenscher, H., Meinl, E., Neipel, F., Mattmann, C., Burns, K., Bodmer, J. L., Schroter, M., Scaffidi, C., Krammer, P. H., Peter, M. E., and Tschopp, J. Viral FLICE-inhibitory proteins (FLIPs) prevent apoptosis induced by death receptors, Nature. 386: 517-21, 1997.

30. Bais, C., Santomasso, B., Coso, O., Arvanitakis, L., Raaka, E. G., Gutkind, J. S., Asch, A. S., Cesarman, E., Gershengorn, M. C., Mesri, E. A., and Gerhengorn, M. C. G-protein-coupled receptor of Kaposi's sarcoma-associated herpesvirus is a viral oncogene and angiogenesis activator [see comments] [published erratum appears in Nature Mar. 12, 1998;392(6672):210], Nature. 391: 86-9, 1998.

31. Yang, T. Y., Chen, S.C., Leach, M. W., Manfra, D., Homey, B., Wiekowski, M., Sullivan, L., Jenh, C. H., Narula, S. K., Chensue, S. W., and Lira, S. A. Transgenic expression of the chemokine receptor encoded by human herpesvirus 8 induces an angioproliferative disease resembling Kaposi's sarcoma, J Exp Med. 191: 445-54, 2000.

32. Leung, D. W., Cachianes, G., Kuang, W. J., Goeddel, D. V., and Ferrara, N. Vascular endothelial growth factor is a secreted angiogenic mitogen, Science. 246: 1306-9, 1989.

33. Flore, O., Rafii, S., Ely, S., O'Leary, J. J., Hyjek, E. M., and Cesarman, E. Transformation of primary human endothelial cells by Kaposi's sarcoma-associated herpesvirus, Nature. 394: 588-92, 1998.

34. Gershengorn, M. C., Geras-Raaka, E., Varma, A., and Clark-Lewis, I. Chemokines activate Kaposi's sarcoma-associated herpesvirus G protein-coupled receptor in mammalian cells in culture [see comments], J Clin Invest. 102: 1469-72, 1998.

35. Brown, Z., Gerritsen, M. E., Carley, W. W., Strieter, R. M., Kunkel, S. L., and Westwick, J. Chemokine gene expression and secretion by cytokine-activated human microvascular endothelial cells. Differential regulation of monocyte chemoattractant protein-1 and interleukin-8 in response to interferon-gamma, Am J Pathol. 145: 913-21, 1994.

36. Koch, A. E., Kronfeld-Harrington, L. B., Szekanecz, Z., Cho, M. M., Haines, G. K., Harlow, L. A., Strieter, R. M., Kunkel, S. L., Massa, M. C., Barr, W. G., and et al. In situ expression of cytokines and cellular adhesion molecules in the skin of patients with systemic sclerosis. Their role in early and late disease, Pathobiology. 61: 239-46, 1993.

37. Standiford, T. J., Stricter, R. M., Kasahara, K., and Kunkel, S. L. Disparate regulation of interleukin 8 gene expression from blood monocytes, endothelial cells, and fibroblasts by interleukin 4, Biochem Biophys Res Commun. 171: 531-6, 1990.

38. Watanabe, K., Kinoshita, S., and Nakagawa, H. Purification and characterization of cytokine-induced neutrophil chemoattractant produced by epithelioid cell line of normal rat kidney (NRK-52E cell), Biochem Biophys Res Commun. 161: 1093-9, 1989.

39. Collins, P. D., Jose, P. J., and Williams, T. J. The sequential generation of neutrophil chemoattractant proteins in acute inflammation in the rabbit in vivo. Relationship between C5a and proteins with the characteristics of IL-8/neutrophil-activating protein 1, J Immunol. 146: 677-84, 1991.

40. Koch, A. E., Polverini, P. J., Kunkel, S. L., Harlow, L. A., DiPietro, L. A., Elner, V. M., Elner, S. G., and Strieter, R. M. Interleukin-8 as a macrophage-derived mediator of angiogenesis [see comments], Science. 258: 1798-801, 1992.

41. Bar-Eli, M. Role of interleukin-8 in tumor growth and metastasis of human melanoma, Pathobiology. 67: 12-8, 1999.

42. Smith, C. W., Chen, Z., Dong, G., Loukinova, E., Pegram, M. Y., Nicholas-Figueroa, L., and Van Waes, C. The host environment promotes the development of primary and metastatic squamous cell carcinomas that constitutively express proinflammatory cytokines IL-1 alpha, IL-6, GM-CSF, and KC, Clin Exp Metastasis. 16: 655-64, 1998.

43. Belperio, J. A., Keane, M. P., Arenberg, D. A., Addison, C. L., Ehlert, J. E., Burdick, M. D., and Strieter, R. M. CXC chemokines in angiogenesis, J Leukoc Biol. 68: 1-8, 2000.

44. Masood, R., Husain, S. R., Rahman, A., and Gill, P. Potentiation of cytotoxicity of Kaposi's sarcoma related to immunodeficiency syndrome (AIDS) by liposome-encapsulated doxorubicin, AIDS Res Hum Retroviruses. 9: 741-6, 1993.

45. Nakamura, S., Salahuddin, S. Z., Biberfeld, P., Ensoli, B., Markham, P. D., Wong-Staal, F., and Gallo, R. C. Kaposi's sarcoma cells: long-term culture with growth factor from retrovirus-infected CD4+T cells, Science. 242: 426-30, 1988.

46. Siegal, B., Levinton-Kriss, S., Schiffer, A., Sayar, J., Engelberg, I., Vonsover, A., Ramon, Y., and Rubinstein, E. Kaposi's sarcoma in immunosuppression. Possibly the result of a dual viral infection, Cancer. 65: 492-8, 1990.

47. Matsushima, K., Morishita, K., Yoshimura, T., Lavu, S., Kobayashi, Y., Lew, W., Appella, E., Kung, H. F., Leonard, E. J., and Oppenheim, J. J. Molecular cloning of a human monocyte-derived neutrophil chemotactic factor (MDNCF) and the induction of MDNCF mRNA by interleukin 1 and tumor necrosis factor, J Exp Med. 167: 1883-93, 1988.

48. Lunardi-Iskandar, Y., Gill, P., Lam, V. H., Zeman, R. A., Michaels, F., Mann, D. L., Reitz, M. S., Jr., Kaplan, M., Berneman, Z. N., Carter, D., and et al. Isolation and characterization of an immortal neoplastic cell line (KS Y-1) from AIDS-associated Kaposi's sarcoma, J Natl Cancer Inst. 87: 974-81, 1995.

49. Marchio, S., Primo, L., Pagano, M., Palestro, G., Albini, A., Veikkola, T., Cascone, I., Alitalo, K., and Bussolino, F. Vascular endothelial growth factor-C stimulates the migration and proliferation of Kaposi's sarcoma cells, J Biol Chem. 274: 27617-22, 1999.

50. Sciacca, F. L., Sturzl, M., Bussolino, F., Sironi, M., Brandstetter, H., Zietz, C., Zhou, D., Matteucci, C., Peri, G., Sozzani, S., and et al. Expression of adhesion molecules, platelet-activating factor, and chemokines by Kaposi's sarcoma cells, J Immunol. 153: 4816-25, 1994.

51. Liu, Z. Y., Ganju, R. K., Yang, Y., Wang, J. F., Gill, P., and Groopman, J. E. Characterization of alpha- and beta-chemokine stimulated signal transdcution pathways in Kaposi' Sarcoma cells, Blood. 594: 135a, 1997.

52. Abruzzo, L. V., Thornton, A. J., Liebert, M., Grossman, H. B., Evanoff, H., Westwick, J., Strieter, R. M., and Kunkel, S. L. Cytokine-induced gene expression of interleukin-8 in human transitional cell carcinomas and renal cell carcinomas, Am J Pathol. 140: 365-73, 1992.

53. Gutman, M., Singh, R. K., Xie, K., Bucana, C. D., and Fidler, I. J. Regulation of interleukin-8 expression in human melanoma cells by the organ environment, Cancer Res. 55: 2470-5, 1995.

54. Munshi, N., Ganju, R. K., Avraham, S., Mesri, E. A., and Groopman, J. E. Kaposi's sarcoma-associated herpesvirus-encoded G protein-coupled receptor activation of c-jun amino-terminal kinase/stress-activated protein kinase and lyn kinase is mediated by related adhesion focal tyrosine kinase/proline-rich tyrosine kinase 2, J Biol Chem. 274: 31863-7, 1999.

55. Liu, Z. Y., Ganju, R. K., Wang, J. F., Ona, M. A., Hatch, W. C., Zheng, T., Avraham, S., Gill, P., and Groopman, J. E. Cytokine signaling through the novel tyrosine kinase RAFTK in Kaposi's sarcoma cells, J Clin Invest. 99: 1798-804, 1997.

56. Venkatakrishnan, G., Salgia, R., and Groopman, J. E. Chemokine receptors CXCR-1/2 activate mitogen-activated protein kinase via the epidermal growth factor receptor in ovarian cancer cells, J Biol Chem. 275: 6868-75, 2000.

57. Arasteh, K. and Hannah, A. The role of vascular endothelial growth factor (VEGF) in AIDS-related Kaposi's sarcoma, Oncologist. 5: 28-31, 2000.

58. Fong, T. A., Shawver, L. K., Sun, L., Tang, C., App, H., Powell, T. J., Kim, Y. H., Schreck, R., Wang, X., Risau, W., Ullrich, A., Hirth, K. P., and McMahon, G. SU5416 is a potent and selective inhibitor of the vascular endothelial growth factor receptor (Fik-1/KDR) that inhibits tyrosine kinase catalysis, tumor vascularization, and growth of multiple tumor types, Cancer Res. 59: 99-106, 1999.

59. Verheul, H. M., Hoekman, K., Jorna, A. S., Smit, E. F., and Pinedo, H. M. Targeting vascular endothelial growth factor blockade: ascites and pleural effusion formation, Oncologist. 5: 45-50, 2000.

What is claimed:

1. An antisense compound 8 to 50 nucleobases in length targeted to a nucleic acid molecule encoding IL-8, wherein said antisense compound comprises at least an 8 nucleobase portion of the sequence 5′-GTT GGC GCA GTG TGG TCC ACT CTC AAT CAC-3′ and wherein said compound inhibits IL-8.

2. The compound of claim 1, wherein said compound is an antisense oligonucleotide.

3. The compound of claim 2, wherein said inhibition is in vitro.

4. The compound of claim 2, wherein said inhibition is in vivo.

5. The compound of claim 2, wherein said antisense oligonucleotide inhibits the proliferation of cultured Kaposi's sarcoma cells at an IC₅₀ of less than or equal to about 5 μM.

6. The compound of claim 2, wherein said antisense oligonucleotide comprises at least one modified internucleoside linkage.

7. The compound of claim 6, wherein the modified internucleoside linkage of the antisense oligonucleotide is a phosphorothioate linkage.

8. A composition comprising the compound of claim 1 and a carrier or diluent.

9. The composition of claim 8, further comprising a colloidal dispersion system.

10. The composition of claim 8, wherein the compound is an antisense oligonucleotide.

11. A method of inhibiting the expression of IL-8 in cells or tissues comprising contacting said cells or tissues with the compound of claim 1 so that expression of IL-8 is inhibited.

12. A method of inhibiting the growth of a Kaposi's sarcoma cell, comprising contacting the cell with an agent effective to inhibit the expression of IL-8 by said cell.

13. The method of claim 12, wherein said agent is selected from the group consisting of the compound of claim 1, an IL-8 antibody, and an agent that inhibits the activity of IL-1β.

14. The method of claim 12, wherein said agent is the compound of claim 1.

15. The method of claim 12, wherein said agent is an IL-1β antibody.

16. The method according to any of claims 13-15, further comprising contacting the cell with a VEGF antagonist.

17. A method of treating Kaposi's sarcoma in an animal, comprising administering to said animal a therapeutically or prophylactically effective amount of the compound of claim 1 so that expression of IL-8 is inhibited.

18. The method of claim 12, wherein said agent is selected from the group consisting of an anti-IL-8 antibody, an IL-8 antisense oligonucleotide and an agent that reduces the concentration of IL-1β in said cell.

19. A method for determining the likelihood of Kaposi's sarcoma in a subject comprising comparing the level of IL-8 obtained from a subject sample to the level of IL-8 in a control sample, whereby a positive diagnosis is made when said level from a subject sample is greater than said control.

20. A method for monitoring the progression of Kaposi's sarcoma in a subject, comprising performing two or more comparisons between the concentration of IL-8 obtained from a suitable subject sample to a control IL-8 concentration, whereby the progression of Kaposi's sarcoma is relative to the concentration of said IL-8 concentration in said subject.

21. The method of claim 20, wherein said comparing is performed by determining the concentration of IL-8 protein in said sample and said control.

22. The method of claim 20, wherein said comparing is performed by determining the concentration of IL-8 mRNA in said sample and said control.

23. The method of any of claims 18 to 21, wherein said sample is a serum sample.

24. A method for determining the likelihood of Kaposi's sarcoma in a subject comprising comparing the level of VEGF obtained from a serum sample of a subject to the level of VEGF of a control, whereby the likelihood of Kaposi's sarcoma is greater when said concentration is greater than said control

25. A method for monitoring the progression of Kaposi's sarcoma in a subject, comprising performing two or more comparisons between the level of VEGF obtained from a serum sample of a subject to the level of VEGF in a control sample, whereby an increase in concentration is associated with progression of Kaposi's sarcoma in said subject.

26. A method for assesssing the risk of Kaposi's sarcoma in a subject comprising comparing the concentration of CRCX1 obtained from the subject sample to a control CRCX1 concentration, whereby a concentration of CRCX1 from the subject sample greater than the control concentration is associated with an increased risk of Kaposi's sarcoma.

27. A method for monitoring the progression of Kaposi's sarcoma in a subject, comprising comparing the concentrations of CRCX1 from at least two samples taken sequentially from a suitable subject, whereby an increase in concentration is associated with progression of Kaposi's sarcoma in said subject.

28. The method of claim 26 or 27, wherein said comparing is performed by determining the concentration of CRCX1 protein in said sample and said control.

29. The method of claim 26 or 27, wherein said comparing is performed by determining the concentration of CRCX1 mRNA in said sample and said control.

30. An isolated polynucleotide consisting essentially of 5′-GTT GGC GCA GTG TGG TCC ACT CTC AAT CAC-3′.

31. An isolated polynucleotide that hybridizes under stringent conditions to the polynucleotide of the sequence 5′-GTT GGC GCA GTG TGG TCC ACT CTC AAT CAC-3′.

32. An isolated polynucleotide that is complementary to the nucleotide sequence 5′-GTT GGC GCA GTG TGG TCC ACT CTC AAT CAC-3′.

33. An isolated polynucleotide that hybridizes under stringent conditions to the polynucleotide of claim 32.

34. A vector comprising a gene encoding an RNA consisting essentially of the sequence 5′-GUU GGC GCA GUG UGG UCC ACU CUC AAU CAC-3′, wherein said gene is operably linked to a promoter capable of expressing said RNA a cell.

35. The vector of claim 34, wherein said cell is a Kaposi's sarcoma cell.

36. A host cell comprising the compound of any of claims 1-7.

37. A host cell comprising the compound of any of claims 30-33.

38. A host cell comprising the vector of claim 34.

39. A kit for detecting or monitoring the progression of Kaposi's sarcoma in a subject comprising an agent that detects the level of an agent selected from the group consisting of IL-8 and CRCX1 and instructions for use.
